(19) United States
(12) Patent Application Publication
(10) Pub. No.: US 2004/0115790 A1
(43) Pub. Date: Jun. 17, 2004

(54) Method for production of secreted proteins in fungi

(76) Inventors: Tiina Pakula, Helsinki (FI); Markku Saloheimo, Helsinki (FI); Jaana Uusitalo, Espoo (FI); Anne Huuskonen, Helsinki (FI); Adrian Watson, Norwich (GB); David Jeenes, Norwich (GB); David Archer, Nottingham (GB); Marja Penttila, Helsinki (FI)

Correspondence Address: SUGHRUE MION, PLLC, 2100 PENNSYLVANIA AVENUE, N.W., SUITE 800, WASHINGTON, DC, 20037, US

(21) Appl. No.: 10/467,710

(22) PCT Filed: Feb. 13, 2002
(86) PCT No.: PCT/FI02/00116

(30) Foreign Application Priority Data
Feb. 13, 2001 (FI) 20010272

(51) Int. Cl. C12N 1/20

(52) U.S. Cl. 435/252.3

(57) ABSTRACT

This invention relates to a promoter and to a fungal host for improved protein production. According to the invention the promoter has been modified in its response to the mechanisms mediating transcriptional down-regulation of secreted proteins under secretion stress. This invention relates also to methods for optimised protein production of secretable proteins in fungi.

This invention relates to an optimised method for the production of secreted proteins in fungi. In particular, this invention relates to DNA sequences, promoters and fungal hosts used in the method.

### BACKGROUND OF THE INVENTION

Certain species of fungi, in particular Trichoderma reesei and Aspergillus niger, are commonly used in biotechnological industry for protein production. The recombinant proteins, either heterologous or homologous, are typically produced under the regulation of promoters of abundantly expressed genes encoding secreted proteins in the fungi, e.g. the promoter of cbh1 of T. reesei and the promoter gla of A. niger. T reesei and A. niger produce homologous hydrolases very efficiently into the culture medium, but the yields of heterologous proteins produced are typically much lower compared to those of homologous proteins. Especially, the proteins originating from distant species e.g. mammalian proteins are produced at a very low level (Archer and Peberdy, 1997, Penttilä, 1998): As reasons for the low yields have been suggested inefficient translation and translocation of the polypeptide into the secretory pathway, hindrances in folding and transport of the protein, and low transcript levels of the heterologous gene due to mRNA instability (MacKenzie et al. 1993, Gouka et al. 1997).

The impairment of protein folding and further transport, likely to occur during production of a heterologous protein, is known to induce stress responses in the cell. In recent years, two feedback mechanisms have been reported that allow the cell to sense the state of the lumen of the ER and respond to perturbations in the normal functioning of this specialised environment for protein folding and processing. These mechanisms constitute the Un-folded Protein Response (UPR), which increases the transcriptional activity of genes encoding chaperones and folding catalysts in response to the presence of unfolded proteins in the lumen of the ER (Shamu et al., 1994) and phosphorylation of the eukaryotic initiation factor 2 α which down-regulates translation activity in the cells (Harding et al., 1999). The cellular response to unfolded proteins in the endoplasmic reticulum of yeast and mammalian cells has been reviewed recently by Mori (2000).

However, very little is known about the transcriptional regulation of genes coding for endogenous secreted proteins in these stress conditions. Especially, no reported data is available on the feed-back regulation of genes encoding the secreted proteins in response to limitations in the capacity of the cell to fold and transport the proteins. In some cases it has been observed that concomitant to the expression of heterologous genes the transcript levels of genes encoding endogenous extracellular proteins are lower compared to the expression in the control strains (Margolles-Clark et al. 1996). The explanation given has been that the amount of transcription/regulatory factors needed for efficient expression of the genes could be limiting during expression of multiple copies of the heterologous gene.

Regulation of the genes encoding secreted proteins on different carbon and nitrogen sources has been studied in detail in filamentous fungi, the cellulase and hemicellulase expression in T. reesei being a good example of that. In T. reesei the cellulase and hemicellulase expression is readily adapted to the environmental, requirements and the availability of nutrients. On complex plant material containing medium, the (hemi)cellulase genes are coordinately induced, but also specific induction mechanisms are known (Margolles-Clark et al. 1997). Cellulose and certain oligosaccharides, such as lactose or sophorose, are known to be efficient inducers of the genes. In the presence of glucose the expression of cellulases and hemicellulases are tightly repressed by the carbon catabolite repression mechanism. Several regulatory factors mediating the regulation of cellulase gene expression have been isolated, including the glucose repressor gene cre1 and as well genes that have been postulated to function as cellulase gene activators (ace1 and ace2) (Saloheimo et al. 2000).

Specifically, modified Trichoderma promoters which are inducible by sophorose and not repressed by the presence of glucose and which comprise a nucleotide sequence from Trichoderma reesei cbh1 promoter upstream of the protein coding region is described in U.S. Pat. No 6,001,595. The publication mentions the regions −184 to −1, −161 to −1, −140 to −1 and −161 to −133. Although the publication describes certain truncated regions of cbh1, it does not suggest their use in the production of secreted proteins under stress conditions. The promoters are designed for protein production in the presence of glucose or sophorose. Cellulase regulators ace1 and ace2 have been described in the International Patent Publication WO 98/23642, which describes their use as activators of protein production and suggests improved hemi(cellulase) expression by overexpression of the factors. Modifications that result in glucose derepression are described in WO 94/04673.

### SUMMARY OF THE INVENTION

This invention is based on the novel finding that the expression level of genes encoding secreted proteins in filamentous fungi is decreased in conditions, in which the protein synthesis, folding or transport is impaired. This regulation mechanism has been demonstrated to function in cultures treated with chemical agents interfering with protein synthesis, folding or transport (DTT, Ca²⁺-ionophore A23187, BrefeldinA, respectively), or in strains with functionally incomplete protein folding system (strains expressing anti-sense transcript for the gene pdiA). In addition, strains producing heterologous proteins, such as tPA (tissue plasminogen activator) have been shown to display activated UPR as well as lower expression levels of endogenous genes coding for secreted proteins. We have as first found that this type of feed-back regulation occurs in the production of secreted proteins in filamentous fungi.

This phenomenon called down-regulation or feed-back regulation of genes encoding secreted proteins, is utilised in this invention to selectively regulate the genes encoding secreted proteins or their promoters, and to enhance production of chosen proteins. This is achieved by genetically modifying the promoter sequence of a gene coding for a secreted protein to alter its responsiveness to the transcriptional down-regulation. Alternatively, the genes coding for the regulatory factors mediating the down-regulation, or factors in the corresponding signalling pathway, can be modified in a way that the down-regulation is either abolished or enhanced. Inactivation of the down-regulation mechanism is beneficial when production of protein of interest takes place under the regulation of a promoter that is normally subjected to down-regulation during secretion stress. Enhancement of the down-regulation can be utilised to repress production of other proteins when the expression of the protein of interest takes place for instance under a promoter that is not subjected to down-regulation.

In this invention has been found that a specific regulatory region or DNA sequence located in a promoter of a secretable protein is capable of mediating transcriptional down-regulation.

More specifically, a DNA sequence located in a promoter of a secretable protein in a fungus is mainly characterized by what is stated in the characterizing part of claim 1.

DNA sequences in the promoter mediating transcriptional down-regulation of secreted proteins under secretion stress can be mutated, inactivated or removed to abolish or reduce the down-regulation of the gene or alternatively the down-regulation of the gene can be enhanced by modifying the promoter sequences, e.g. by amplifying the responsive promoter element, subjected to the down-regulation.

More specifically a promoter of a secretable protein is mainly characterized by what is stated in the characterizing part of claim 6.

A method for producing a promoter for improved protein production in a fungal host is stated in the characterizing part of claim 10.

The invention can be used to design better strains for protein production by increasing the efficiency of the promoters used for protein production and/or manipulating the regulation system of secreted proteins.

More specifically a fungal host strain for optimised protein production is mainly characterized by what is stated in the characterizing part of claim 12.

A method for producing a fungal host for improved protein production is mainly characterized by what is stated in the characterizing part of claims 26 and 27.

The present invention can be used for modification of a homologous or heterologous promoter used for production of a protein, either homologous or heterologous, in a way that the expression is not subject to the down-regulation in a similar manner as the unmodified promoter. In particular, the invention is useful for producing heterologous proteins but can be applied also to production of homologous proteins.

Furthermore the invention can be used for inactivation or reducing the activity or expression of the regulatory factor(s) mediating the down-regulation of the promoter to improve protein production under the promoter, either the regulatory factor(s) binding to the promoter or regulatory factors mediating the response.

A method for optimised protein production of secretable proteins in fungi is mainly characterized by what is stated in the characterizing part of claims 28 and 30.

One possibility to use the invention is overexpression of a regulatory factor to decrease production of homologous secreted proteins during expression of homologous or heterologous proteins under a promoter that is not subjected to the down-regulation. In such case a heterologous protein is expressed and secreted e.g. under a promoter such as Trichoderma gpd, which is not affected in stress conditions. Homologous secreted proteins are expressed under a promoter which is down-regulated. Genes encoding proteins mediating the down-regulation are overexpressed.

A method for optimised protein production of secretable proteins in fungi is mainly characterized also by what is stated in the characterizing part of claim 31.

Use of the DNA sequence, promoter or fungal host prepared according to this invention is characterized by what is stated in claim 31.

Other features, aspects and advantages of the present invention will become apparent from the following description and appended claims.

### FIGURES

FIG. 1. Total protein synthesis and secretion in cultures treated with A23187, DTT and BFA.

A) The amount of radioactivity incorporated into TCA insoluble material in cell extracts prepared from cultures treated with 5 uM A23187 (open diamonds, ⋄), and from non-treated control cultures (black diamonds, ♦).

B) The amount of radioactivity incorporated into TCA insoluble material in culture supernatant in cultures treated with 5 uM A23187 (open diamonds, ⋄), and from non-treated control cultures (black diamonds, ♦).

C) The amount of radioactivity incorporated into TCA insoluble material in cell extracts prepared from cultures treated with 10 mM DTT (open diamonds, ⋄), and from non-treated control cultures (black diamonds, ♦).

D) The amount of radioactivity incorporated into TCA insoluble material in culture supernatant in cultures treated with 10 mM DTT (open diamonds, ⋄), and from non-treated control cultures (black diamonds, ♦).

E) The amount of radioactivity incorporated into TCA insoluble material in cell extracts prepared from cultures treated with 50 μg/ml BFA (open diamonds, ⋄), and from non-treated control cultures (black diamonds, ♦).

F) The amount of radioactivity incorporated into TCA insoluble material in culture supernatant in cultures treated with 50 μg/ml BFA (open diamonds, ⋄), and from non-treated control cultures (black diamonds, ♦).

FIG. 2. 2D gel analysis of CBHI from cultures treated either with 5 μM A23187, 50 μg/ml BFA or 10 mM DTT and labelled with ³⁵S-methionine for different time periods.

A) Labelled CBHI from cell extracts prepared from non-treated control culture and from cultures treated with A23187, DTT or BFA at different time points during the labelling experiment (the time point is indicated above each panel as minutes after addition of the labelled methionine).

B) Labelled CBHI from culture supernatant after 180 minutes of labelling of the cultures treated with A23187 or BFA and of non-treated cultures.

FIG. 3. The synthesis and secretion of CBHI.

A) The amount of labelled CBHI in the cell extract at different time points of the labelling experiment in cultures treated with 5 μM A23187 (open circles, ∘) and in non-treated control cultures (black diamonds, ♦)

B) The amount of labelled CBHI in the culture supernatant at different time points of the labelling experiment in cultures treated with 5 μM A23187 (open circles, ∘) and in non-treated control cultures (black diamonds, ♦)

C) The amount of labelled CBHI in the cell extract at different time points of the labelling experiment in cultures treated with 10 mM DTT (open circles, ∘) and in non-treated control cultures (black diamonds, ♦)

D) The amount of labelled CBHI in the culture supernatant at different time points of the labelling experiment in cultures treated with 10 mM DTT (open circles, ∘) and in non-treated control cultures (black diamonds, ♦)

E) The amount of labelled CBHI in the cell extract at different time points of the labelling experiment in cultures treated with 50 μg/ml BFA (open circles, ∘) and in non-treated control cultures (black diamonds, ♦)

F) The amount of labelled CBHI in the culture supernatant at different time points of the labelling experiment in cultures treated with 50 μg/ml BFA (open circles, ∘) and in non-treated control cultures (black diamonds, ♦)

FIG. 4. Northern analysis of pdi1 and bip1 expression in cultures treated with A23187, DTT or BFA.

A) The steady-state mRNA level of bip1 and pdi1 (the signals normalised with those of gpd) at different time points of the treatment with A23187 (the black bars) and in non-treated control cultures (the white bars)

B) The steady-state mRNA levels of bip1 and pdi1 (the signals normalised with those of gpd) at different time points of the treatment with with DTT (the black bars) and in non-treated control cultures (the white bars)

C) The steady-state mRNA levels of bip1 and pdi1 (the signals normalised with those of gpd) at different time points of the treatment with with BFA (the black bars) and in non-treated control cultures (the white bars)

FIG. 5. Northern analysis of hac1 mRNA in cultures treated with BFA and A23187 (the black bars) and in the non-treated control cultures (the white bars).

FIG. 6. Northern analysis of cbh1 and egl1 in cultures treated with A23187, DTT or BFA for different time periods (the black bars) and in the control cultures (the white bars).

FIG. 7. Northern analysis of xyn1 and hfb2 proteins in T. reesei during DTT treatment (signals normalised with the signal of gpd)

FIG. 8. Northern analysis of transcripts that are not down-regulated during treatment with DTT: signals of ypt1 and sar1 coding for components of the secretory pathway, cDNA1 of unknown function, and bgl2 coding for intracellular β-glucosidase (the (signals were normalised with the signal of gpd)

FIG. 9. The effects of DTT on the transcription of genes from A. niger:

A) the glucoamylase gene, glaA (average signal of three determinations)

B) the acid protease, aspergillopepsin gene (pepA)(average signal of three determinations)

C) protein disulfide isomerase (pdiA), a foldase resident in the ER, (average signal of three determinations)

D) bipA, an ER-resident chaperone, (average signal of three determinations) (a solid line representing the DTT-treated cultures and a dotted line representing the water treated controls)

FIG. 10. The effect of exchanging medium containing starch as a carbon source for medium containing xylose as a carbon source on the transcription of glaA in A. niger AB4.1 (The exchange was accomplished at T=0., and the results represent the average of two determinations)

FIG. 11.

A) The effect of antisense pdiA on the transcription of the glucoamylase gene (glaA, average signal of six flasks from two different experiments) (The strain AB4.1 is represented by a solid line, and the strain AS1.1 by a dotted line.)

B) The effect of antisense pdiA on the transcription of the aspergillopepsin gene (pepA, average signal of six flasks from two different experiments). (The strain AB4.1 is represented by a solid line, and the strain AS1.1 by a dotted line.)

C) The dry weight determination for the cultures (average signal of six flasks from two different experiments)

FIG. 12.

A) Levels of secreted glucoamylase protein for A. niger expressing antisense pdiA under the control of the gpdA promoter. (The data are averages of three determinations for each time-point, the strain AB4.1 is represented by black bars, and the strain ASG67 by a grey bars).

B) Steady-state levels of glaA mRNA in A. niger expressing antisense pdiA under the control of the gpdA promoter. (The strain AB4.1 is represented by a solid line, and the strain ASG67 by a dotted line).

C) The dry weight determination for the cultures. (The strain AB4.1 is represented by a solid line, and the strain ASG67 by a dotted line).

FIG. 13. Northern blot analysis of A. niger AB4.1 and ASG67 probed with hacA. Lanes 1-7 show samples for A. niger AB4.1 at 24, 36, 48, 60, 72, 84 and 96 hours while lanes 8-14 shown the same time-points for A. niger ASG67 (antisense pdiA strain).

FIG. 14. Bioreactor cultivation of T. reesei Rut-C30 and a tPA producing transformant 306/36

A) The expression cassette for production of tPA in T. reesei Rut-C30

B) Biomass dry weight and lactose concentration measured during the cultivation to monitor growth.

C) Total protein and HEC activity (measuring cellulase, especially endoglucanase, activity) produced in the culture medium.

D) Transcript level of egl1 (normalised with the signal of actin gene) analysed as an example of a gene coding for a secreted protein.

E) Transcript level of cbh1 and cbh1-tPA fusion (normalised with the signal of actin gene

F) Transcript level of bip1 (normalised with the signal of actin gene) during the cultivation

FIG. 15. Expression of the lacZ reporter gene under cbh1 promoter

A) Schematic view of the lacZ expression cassettes used for the expression studies in the presence of 10 mM DTT: the lacZ expression under the full-length cbh1 promoter in the strain pMLO16, and under the shortened promoter in. the strain pMI34.

B) The mRNA levels of lacZ and egl1 (normalised with gpd signal) during the treatment with 10 mM DTT (the black bars) and in the non-treated control cultures (the white bars) in the strain pMLO16 (the graphs on the left) and in the strain pMI34 (the graphs on the right).

FIG. 16. Expression of the lacZ reporter gene under cbh1 promoters deleted to different extent

A) Schematic presentation of the deletion series of cbh1 promoter used for the E. coli lacZ expression in T. reesei for studying the the activity of the promoter under the secretion stress conditions

B) Northern analysis of the expression of lacZ, egl1 and gpd1 in DTT treated cultures of the strains pMLO16, pMLO16S, del1(1)S and del0(2)S and in untreated cultures of the same strains. The time point of the treatment is indicated above each lane as minutes from the onset of the treatment. The relative mRNA levels in the DTT treated cultures at each time point (the mRNA level in the untreated control culture was set as 1) are shown in the graphs on the right (lacZ signal, the open circles; egl1 signal, the black diamonds)

C) Northern analysis of the strains del23, del5(11)S, del5(11) and del6(14) as in B)

D) Northern analysis of he strains del7(5)S, pMI33 and pMI34 as in B)

FIG. 17. The cbh1 mRNA level during DTT treatment in cultures of T. reesei QM9414 and QM9414 with a deleted ace1 gene

FIG. 18. Screening of fungal mutants defective in the mechanism of down-regulation of genes under secretion stress conditions

A) Microtiter plate cultures of the strains pMLO16, pMI33 and QM9414 tested for lacZ production in the presence and absence of BFA and sophorose. LacZ production was detected as a darker color reaction

B) Screening of mutants in microtiter plate cultures for the expression of lacZ after sophorose induction in the presence of BFA. LacZ producing mutants were detected based on the dark color color reaction after addition of X-gal substrate. The cultures of unmutagenised pMLO16 were were assayed for lacZ production after sophorose induction in the presence and absence of BFA mutants (the wells indicated by the boxes)

### DETAILED DESCRIPTION OF THE INVENTION

By the term “endogenous proteins” is meant here proteins which are natural products of a microorganism host.

By “recombinant proteins” are meant here proteins that are not natural products of a microorganism. DNA sequences encoding desired homologous or heterologous proteins may be transferred by a suitable method to a host. By “homologous protein” is meant a protein produced by the same microorganism species. By “heterologous protein” is meant a protein produced by another microorganism species.

By “secretable protein” or “secreted protein” is meant here a protein that is secreted outside of the host cell to the culture medium.

By “improved protein production” is meant protein production which is at least 3%, preferably at least 5%, more preferably at least 10%, still more preferably at least 20%, most preferably at least 30% better than protein production by using a fungal host strain which has not been genetically modified to alter their down-regulation.

By “secretion stress” or “secretion stress conditions” we mean here that the secretion capacity of the host is limited or the secretion route overloaded. The limitation may be caused for example by the production of heterologous proteins or increased amounts of homologous proteins, or it may be due to a toxin hindering the synthesis, folding or transport of the protein (e.g. ionophore, DTT or brefeldin A (=BFA)). The limitation can be caused also by modification of the folding or secretion route by genetical means e.g. by enhancing or inactivating the activity of the components required for protein folding or transport. Like UPR, this mechanism of transcriptional down-regulation of secreted protein genes can, however, be considered a natural mechanism for the organism to balance the synthesis of secreted proteins and the folding and secretion capacity, and may (partially) occur in many protein production conditions, such as when the synthesis of secreted homologous proteins is induced.

By “down-regulation”, “transcriptional down-regulation” or “feed-back-regulation” we mean here that the mRNA levels corresponding to a protein are lowered due to these cellular responses mentioned above. This down-regulation effect has been shown by measuring the mRNA level of the genes encoding secreted proteins.

According to this invention, DNA sequences mediating down-regulation of genes encoding secreted proteins can be found in the promoters of the genes coding for secretable proteins. This means that the promoters comprise regions which are able to down-regulate the gene product, as a response to the action of cellular mechanisms such as regulatory factors.

Various conditions can be used for analysing down-regulation of a gene under secretion stress, as described above. These include e.g. conditions under which secreted proteins are overproduced (heterologous or endogenous proteins), or using toxins, like DTT, BFA or Ca-ionophore, or by modification of the folding or secretion route by genetical means, e.g. by enhancing or inactivating the activity of the components required for protein folding or transport. In this invention the secretion stress was simulated by treating the cultures with DTT, Ca-ionophore A23187, and BFA, or expressing a heterologous secreted protein (tissue plasminogen activator), or reducing by genetic modification the activity of the folding machinery (using the anti-sense technique).

For the purposes of this invention a promoter is defined to comprise DNA sequences mediating transcriptional down-regulation (or down-regulation) if the amount mRNA obtained under the control of the promoter is lower when the host comprising the promoter is grown under secretion stress conditions (as described above) compared with the mRNA amount obtained when the host is grown under non-secretion stress conditions.

In a similar manner for the purposes of this invention a host is defined to comprise mechanisms such as regulatory factors mediating transcriptional down-regulation or down-regulation if the mRNA amount of a gene or genes encoding secreted protein(s) is lower when the host is grown under secretion stress conditions (as described above) compared with the mRNA amount when the host is grown under non-secretion stress conditions.

The down-regulation effect is shown by measuring the mRNA level of the gene under secretion stress as described above. For the purposes of this invention a promoter or host is genetically modified in its response to mechanisms mediating transcriptional down-regulation, if a measurable change can be shown in the mRNA level of the genes encoding secreted protein (s). In other words the expression (mRNA amount) of a selected secretable protein is enhanced or decreased. Preferably the change is 10% or more, more preferably 20% or more, still more preferably 30% or more, most preferably 50% or more, increase or decrease compared to a non-modified promoter or host.

“A reporter protein” is for the purposes of this invention any gene or protein the expression or amount of which can be analyzed. When testing the capacity of a promoter or a host in mediating transcriptional down-regulation under secretion stress as described above, the mRNA level of the reporter protein can be analyzed.

The DNA sequences or regions mediating down-regulation of secreted proteins are located in the promoters of various genes coding for proteins such as cellulases, hemicellulases, amylolytic enzymes, hydrophobins, proteases, invertases, phytases, phosphatases, swollenins, and pectinases.

Preferably the DNA sequences are located in the promoters selected from the group comprising cbh1, cbh2, egl1,egl2, hfb1, hfb2, xyn1, swo, gla, amy, and pepA promoters.

This invention shows that a number of genes encoding sectered proteins by Trichoderma and Aspergillus are subject to the transcriptional down-regulation mechanism. The promoter of a secretable protein according to this invention is preferably a promoter of an efficiently secreted hydrolase of the genus Trichoderma. More preferably the promoter is a cellulase or hemicellulase promoter of Trichoderma. Most preferably the promoter is cbh1 of Trichoderma. The promoter of a secretable protein may also be the promoter of an efficiently secreted hydrolase of the genus Aspergillus. Preferably the promoter is a protease or a promoter of an amylolytic enzyme gene of Aspergillus. More preferably the promoter is gla, amy or pepA.

As exemplified in this invention DNA sequences mediating down-regulation of secretable proteins can be found in Trichoderma cbh1 promoter upstream of −162 (SEQ ID NO. 5). Alternatively they can be found upstream of −188 (SEQ ID NO. 2), −211(SEQ ID NO. 3), −341(SEQ ID NO. 4), −391(SEQ ID NO. 1), −501(SEQ ID NO. 8), −741(SEQ ID NO. 9), −881(SEQ ID NO. 10). However, they seem to be located downstream of −1031 (SEQ ID NO. 11), −1201 (SEQ ID NO. 7) or −1281 (SEQ ID NO. 6). The DNA sequences mediating down-regulation of secretable proteins in cbh1 promoter seem therefore to be located between the nucleotides −1031 and −162. The most important area being between −211 and −341 and between the nucleotides −501 and −1031.

According to one embodiment of this invention a promoter of a secretable protein is genetically modified not to be down-regulated or reduced in down-regulation.

In a promoter modified according to this invention the effect of the DNA sequences mediating down-regulation of secreted proteins is diminished by various mutation methods, or the sequences may be inactivated or removed. For example, promoters where the DNA sequence mediating down-regulation of secretable proteins is deleted are promoters lacking the nucleotides upstream of −501 (SEQ ID NO. 16), −188 (SEQ ID NO. 17), −211 (SEQ ID NO. 18), −341 (SEQ ID NO. 119), −391 (SEQ ID NO. 20), −162 (SEQ ID NO. 21), −881 (SEQ ID NO. 22) and −741 (SEQ ID NO. 23) of Trichoderma cbh1 promoter.

In another promoter modified according to this invention the effect of the DNA sequences mediating down-regulation of secreted proteins may be increased by amplifying the sequence responsible for mediating the down-regulation using standard molecular biology methods.

For optimised production of secretable proteins fungal host strains may be constructed, in which mechanisms that down-regulate transcription of genes encoding secreted proteins under secretion stress have been genetically modified.

According to one embodiment of this invention the fungal host strain of this invention may comprise a promoter in which the effect of the DNA sequences mediating down-regulation of secreted proteins is diminished or removed or the effect of the DNA sequences mediating down-regulation of secreted proteins is increased.

According to another embodiment of this invention the expression of the regulatory factors mediating transcriptional down-regulation may be genetically modified in the fungal host. If desired, the expression of the regulatory factors may be reduced or abolished, or the expression of the regulatory factors may be increased.

This invention shows that a number of genes encoding extracellular secreted proteins are transcriptionally down-regulated in the conditions used to demonstrate this regulatory mechanisms and it is expected that many, if not most or all, genes encoding secreted proteins are subject to this transcriptional down-regulation. The genes, promoters and proteins subject to the regulatory mechanism may be selected from the group comprising cellulases (such as cellobiohydrolases, endoglucanases and β-glucosidases), hemicellulases (such as xylanases, mannases, β-xylosidases, and side chain cleaving enzymes, such as arabinosidases, glucuronidases, acetyl xylan esterases), amylolytic enzymes (such as α-amylases, glucoamylases, pullulanases, cyclodextrinases) hydrophobins, proteases (acidic, alkaline, aspergillopepsin), invertases, fytases, phosphatases, various pectinases (such as endo-and exopolygalacturonases, pectin esterases, pectin and pectin acid lyase) and ligninases (such as lignin peroxidases, Mn peroxidases, laccases).

The regulatory mechanisms are mediating transcriptional down-regulation of the proteins selected from the group comprising those encoded by the genes cbh1, cbh2, egl1,egl2, hfb1, hfb2, xyn1, swo, gla, amy, and pepA.

As an example the regulatory factor is encoded by the ace1 gene. Other factors than ace1 are also involved in this down-regulation which is shown in the examples by the fact that ace1 is not responsible for (the major part) of the regulation in all culture conditions.

The regulatory mechanism are preferably regulating the hydrolases of the genus Trichoderma. More preferably they are regulating cellulases or hemicellulases of Trichoderma. The regulatory factors may be regulating the hydrolases of the genus Aspergillus. Preferably they are regulating proteases or amylolytic enzymes of Aspergillus.

“A fungal production host” denotes here any fungal host strain selected or genetically modified to produce efficiently a desired product and is useful for protein production for e.g. analytical, medical or industrial use. The host strain is preferably a recombinant strain modified by gene technological means to efficiently produce a product of interest.

The invention is here exemplified by two fungal species Trichoderma and Aspergillus, which shows the general nature of the transcriptional down-regulation mechanism. Modification of this mechanism in other fungi will be useful for improved protein production.

Fungal host strains of this invention can be selected from the group comprising Aspergillus spp., Trichoderma ssp., Neurospora spp., Fusarium ssp., Penicillium ssp., Humicola ssp., Tolypocladium geodes, Schwanniomyces ssp., Arxula ssp., Trichosporon ssp., Kluyveromyces ssp., Pichia ssp., Hansenula ssp., Candida spp., Yarrowia ssp, Schizosaccharomyces ssp. and Saccharomyces ssp. Preferably the host belongs to Trichoderma or Aspergillus species, e.g. T. harzianum, T. longibrachiatum, T viride, T. koningii, A. nidulans, A. terreus, A. ficum, A. oryzae and A. awamori. Most preferably it belongs to T. reesei (Hypocrea jecorina) or A. niger species.

A method for optimised protein production of secretable proteins in fungi comprises the steps of:

selecting a gene encoding a secretable protein;

genetically modifying the promoter of the gene in its response to mechanisms mediating transcriptional down-regulation of secreted proteins under secretion stress;

producing a desired secretable protein under the regulation of the promoter in a fungal host; and

recovering the protein product from the culture medium of the host.

According to this invention a method for optimised protein production of secretable proteins in fungi may comprise the steps of:

cultivating a fungal host as defined above in a suitable culture medium; and

recovering the protein product from the medium.

The protein product may be any product originating from bacteria or higher or lower eukaryotes, the protein product may originate from fungal or mammalian origin. The protein product may be a hydrolase, such as cellulase, hemicellulase, amylolytic enzyme, hydrophobin, protease, invertase, fytase, phosphatase, pectinase or it may be any mammalian protein, such as immunoglobulin or tPA.

According to one embodiment of this invention the protein product may be expressed from a promoter not subject to transcriptional down-regulation. Other, undesired proteins may be expressed from a promoter regulated by down-regulation. By enhancing the down-regulation, it is possible to direct the production to the protein product expressed from a promoter not subject to transcriptional down-regulation. Such promoter may be a constitutive promoter, such as gpd.

A method for optimised protein production of secretable proteins in fungi comprises the steps of:

selecting a gene of a secretable protein;

operable linking the coding region of the selected secretable protein into a promoter not regulatable by the mechanism of transcriptional down-regulation;

culturing the fungal host under suitable culture conditions and overproducing proteins mediating down-regulation in the fungal host; and

recovering the selected secretable protein from the culture medium of the host.

The selected secretable protein may be a heterologous protein and the undesired secretable proteins may be homologous proteins.

By“genetically modifying the promoter to be or not to be regulatable by down-regulation” means here that the promoter has been modified by any suitable conventional or molecular biology method well known in the art to be or not to be regulated by down-regulation in a similar manner than the unmodified promoter is, by DNA techniques, such as by site directed mutagenesis or deletion, or by conventional mutagenesis using chemical agents or irradiation, followed by screening or selecting for cells modified in the transcriptional down-regulation mechanism. In this invention the genetic modification has been exemplified by deleting parts of Trichoderma cbh1 promoter not to be regulated by down-regulation.

“Genetically modifying the genes encoding proteins mediating down-regulation in secretion stress” means here that the genes have been modified by any suitable conventional or molecular biology method well known in the art to be overproduced or inactivated or modified in their activity or expression. The modification is preferably made by recombinant DNA techniques, such as by site directed mutagenesis or deletion but also any other method for genetic modification can be used, such as crossing or fusing cells with desired properties, or by conventional mutagenesis using chemical agents or irradiation, followed by screening or selecting for cells modified in the transcriptional down-regulation mechanism.

We have demonstrated the existence of down-regulation mechanism of genes coding for the secreted proteins in filamentous fungi in response to secretion stress. Examples are shown from the fungal species T. reesei and A. niger. Evidence. for the novel regulation mechanism has been obtained analysing fungal cultures treated with chemical agents preventing either protein synthesis, folding or transport, or by analysing fungal strains displaying diminished foldase levels (see Examples 1, 2, 3, 4 and 5). In addition, in strains producing heterologous proteins, the genes coding for endogenous secreted proteins are expressed at lower levels compared to their parental strain (see Example 6). In eukaryotic systems two feedback mechanisms have been reported in recent years that allow the cell to sense the state of the lumen of the ER and respond to secretion stress to alleviate the perturbations. These include the UPR pathway (Shamu et al., 1994) and attenuation of translation initiation (Harding et al., 1999). Our novel finding comprises a third type of feed-back regulation mechanism functioning under secretion stress, which is shown to be mediated by the promoter sequence of the particular gene using a reporter gene system consisting of lacZ expression under cbh1 promoter sequences (Examples 7 and 8).

Based on the results obtained it is possible to continue characterization of the promoter regions involved in the down-regulation of the genes coding for secreted proteins in Trichoderma. The promoter regions involved can be localised by studying for example the lacZ reporter gene expression under the cbh1 promoter that has been deleted to different extent and using conditions in which the mRNA level of the genes coding for extracellular proteins is down-regulated, e.g. treatment with DTT. Based on this analysis, selected promoter regions can be used in gel shift assays with cell extracts from stressed and non-stressed cultures (e.g. DTT-treated and non-treated) to identify the specific regions even more in detail, and to characterize possible binding sites for regulatory factors. Comparison of the promoter sequences can be used for identification of sequences mediating the down-regulation in other promoters that are affected in the stress conditions. Using the methods described here or known in the art it is possible to identify regions in any organism and any promoter from a gene encoding a secreted protein, responding to this transcriptional down-regulation.

Cloning and characterization of the regulatory proteins involved in the feed-back regulation and binding to the promoter sequences can be performed using e.g. yeast-one hybrid system taking advantage of the characterised promoter elements in the cbh1 promoter (and in the other relevant genes showing down-regulation). Cloning systems for DNA binding proteins that can be applied are commercially available (e.g. Matchmaker™ by Clontech) or have been reported (e.g. Saloheimo et al. 2000).

The promoter sequence found to be mediating the down-regulation of the gene can be modified in such a way that the down-regulation in stress conditions is abolished or reduced. By these means it is possible to increase the production level of the gene in conditions where it would otherwise be down-regulated, and production of either an homologous or a heterologous gene product under the modified promoter (from which the down-regulating sequences have been modified) can be improved. In addition the regulatory factors involved in the down-regulation can be completely or partially inactivated to improve protein production. Similar approach can be taken with any organism known to possess down-regulation of genes coding for secreted proteins, e.g. other species of fungi, preferably other species of filamentous fungi.

Production of heterologous proteins may cause similar type of stress response as e.g. the treatment with the chemical agents DTT, BFA or A23187. Lower levels of endogenous cellulase transcripts have been observed in T. reesei cultures producing human tissue plasminogen activator indicating down-regulation of the genes coding e.g. for egl1 and cbh1 during production of tPA (Example 6). If the promoters of the genes coding for the endogenous extracellular proteins are used for expression of the heterologous product or overexpression of a homologous product inducing stress responses, the expression may become subject to the feed-back regulation mediating transcriptional down-regulation during the production. Modification of either the promoter elements or the regulatory factors binding to the promoter or mediating the regulatory signal are means to increase protein production by abolishing the down-regulation process.

In some cases it may it may be beneficial to enhance down-regulation during secretion stress to diminish production of some of the endogenous proteins, and to produce the protein of interest under a promoter that is not down-regulated during secretion stress. This can be achieved by overexpressing the regulatory factors mediating the down-regulation and/or by modifying the promoters to increase the binding of the repressing regulatory factors, e.g. increasing the number of binding sites for the factors. This invention describes one method how those genes can be identified whose promoters are not down-regulated when the expression of secreted protein genes are, an example being the T.reesei gpd promoter.

By these means it is possible to selectively regulate the genes coding for secreted proteins to enhance the production of chosen proteins, either by reducing or inactivating the down-regulation of the production promoter or by enhancing down-regulation to selectively repress expression of other secreted proteins. It is to be noted that the invention can be utilised not only in protein production but that the mechanisms of transcriptional down-regulation described here provides means to modify fungal strains also for other purposes and selectively regulate the expression of certain undesired or desired proteins in the host.

### EXAMPLES

### Example 1

### The Effects of the Ca²⁺-Ionophore A23187, Dithiothreitol (DTT) and Brefeldin A (BFA) in T. reesei Rut-C30 Cultures

Trichoderma Strains, Cultivation Conditions and Methods Used for Sampling, Metabolic Labelling and Analysis of RNA and Proteins.

The Trichoderma strains and cultivation conditions have been essentially described elsewhere (Pakula et al. 2000; Ilmén et al 1996). T. reesei strain Rut-C30 (Montenecourt & Eveleigh, 1979) was cultivated on minimal medium ((H₄)₂SO₄ 7.6 g l⁻¹, KH₂PO₄ 15.0 g l⁻¹, MgSO₄.7H₂O 0.5 g l⁻¹, CaCl₂.H₂O 0.2 g l⁻¹, CoCl₂ 3.7 mg l⁻¹, FeSO₄.7H₂O 5 mg l⁻¹, ZnSO₄.7H₂O 1.4 mg l⁻¹, MnSO₄.7H₂O 1.6 mg l⁻¹, pH adjusted to 5.2 with KOH) that contained lactose 20 g l⁻¹ as a carbon source. Spore suspension with 2×10⁷ spores (stored in −80° C. in 20% glycerol) was inoculated into 200 ml of the medium, grown in shake flasks at 28° C. with shaking at 210 rpm. After 4 days of cultivation the cultures were diluted 1/10 into fresh medium, grown further for 24 h, and treated either with 10 mM dithiothreitol (DTT), 50 μg/ml brefeldin A (BFA) or 5 μM Ca²⁺-ionophore A23187. A corresponding volume of the solvent of the stock solution was added into the untreated control cultures (0.2% and 0.5% DMSO for the control cultures for A23187 and BFA treatment, respectively, and double distilled water for the control for DTT treatment). The cultures were divided into aliquots for metabolic labelling of the proteins and for RNA isolation at different time points.

Proteins were metabolically labelled with ³⁵S-methionine using the methods described in (Pakula et al. 2000). The preparation of the samples and analysis of the labelled proteins were carried out essentially as in (Pakula et al. 2000). The labelling experiment was started after 10 min of addition of DTT or A23187 or after 15 min of addition of BFA. 1 mCi of [³⁵S]-methionine (Amersham S J 1015, in vivo cell labelling grade, 1000 Ci mmol⁻¹, 10 μCi μl⁻¹) was added to a 50 ml aliquot of the cultivation. Untreated cultures were labelled in parallel and in a similar manner. Samples of 2 ml were collected during a time course. Labelled total protein in cell extracts and culture supernatant was measured using scintillation counting of TCA insoluble material in the samples, and labelled specific proteins (e.g. CBHI) were analysed using 2D gel electrophoresis, and the proteins were quantified using a phosphorimager (Molecular Dynamics). The rates of protein synthesis and secretion, and the average synthesis time and minimum secretion time were determined as described in Pakula et al. 2000.

For the Northern analysis mycelial samples were collected from cultures treated with DTT, BFA or A23187 and from the untreated control cultures after 0, 15, 30, 60, 90, 120, 240 and 360 minutes of the treatment. The first sample (the time point 0 min was withdrawn immediately before addition of DTT, BFA or A23187). The mycelium was filtered, washed with equal volume of 0.7% NaCl, frozen immediately in liquid nitrogen, and stored at −80° C. Total RNA was isolated using the Trizol™ Reagent (Gibco BRL) essentially according to manufacturer's instructions. Northern blotting and hybridisation was carried out according to standard procedures (Sambrook et al). Full-length cDNA of the genes were used as probes.

The Effect of A23187, DTT and BFA on Protein Synthesis and Transport in T. reesei

Feedback regulation of genes coding for secreted proteins was studied in cultures treated with reagents known to interfere either with protein synthesis, folding or transport in other organisms. The Ca²⁺-ionophore A23187 has been reported to reduce protein synthesis as well as to inhibit protein folding and transport by emptying the Ca²⁺ stores of the ER in mammalian cells (Broström et al. 1989, Lodish and Kong, 1990, Lodish et al. 1992). Dithiothreitol is a reducing agent that inhibits formation of the disulphide bridges and protein folding in yeast and in mammals (Jämsä et al. 1994, Alberini et al. 1990, Braakman et al. 1992). Treatment of the cells with BFA is known to disrupt Golgi structure and inhibit transport of proteins from the ER to Golgi e.g. in mammalian systems, but the effect is dependent on the organism and the specific cell type (Pelham, 1991, Shah and Klausner, 1993).

Metabolic labelling of the proteins was used to characterise the effects of A23187, DTT and BFA on protein synthesis and secretion in T. reesei cultures (see Pakula et al. 2000 for the methods used).

The cultures were treated for 10 minutes with 5 μM A23187 or 10 mM DTT or for 15 minutes with 50 μg/ml BFA before the addition of the labelled methionine. Labelled total protein as well as labelled specific proteins were analysed in cell extract and culture supernatant at different time points of the labelling experiment.

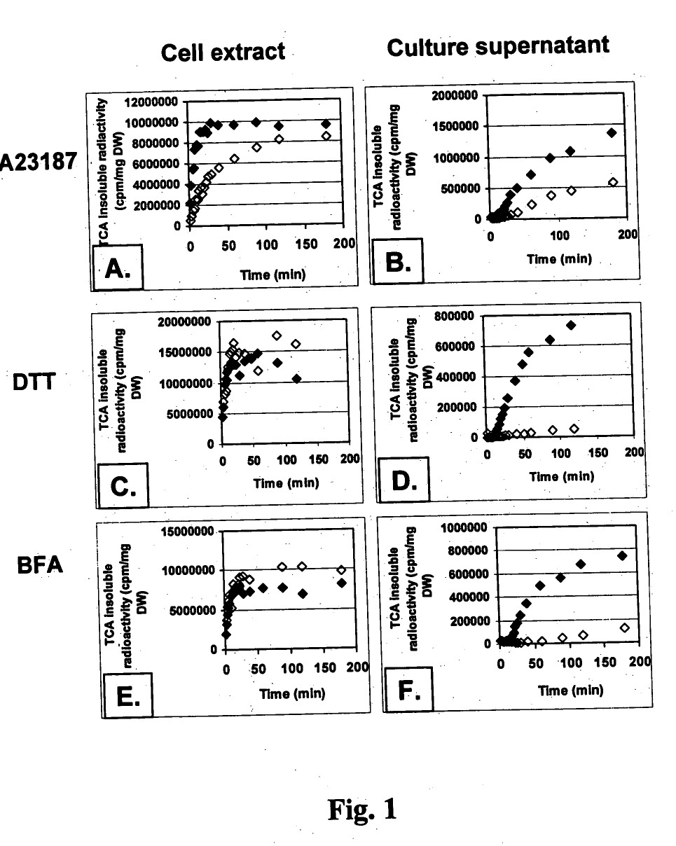

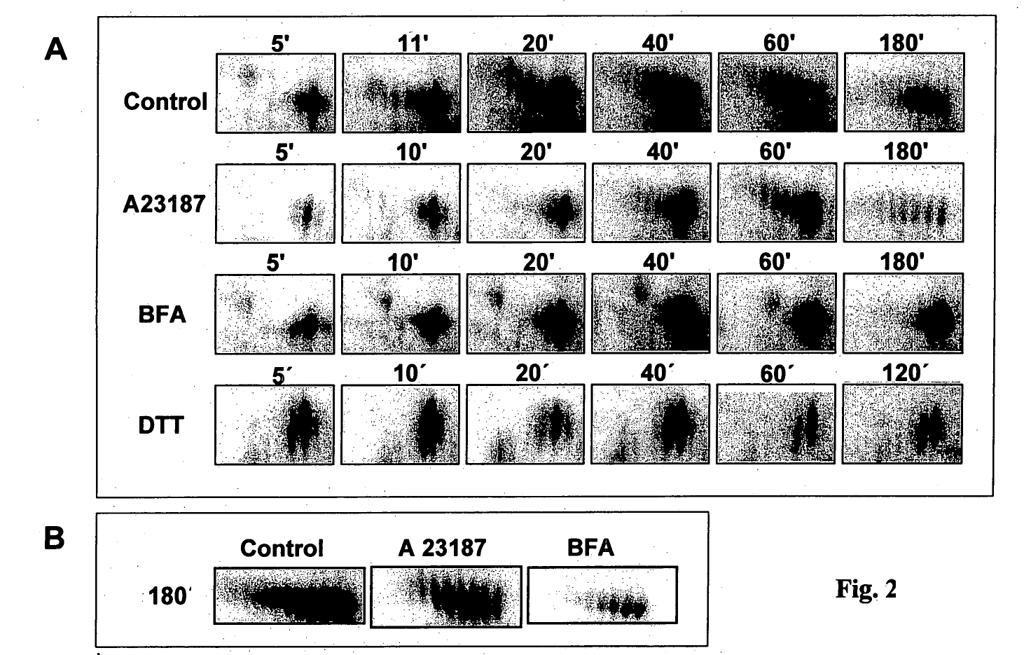

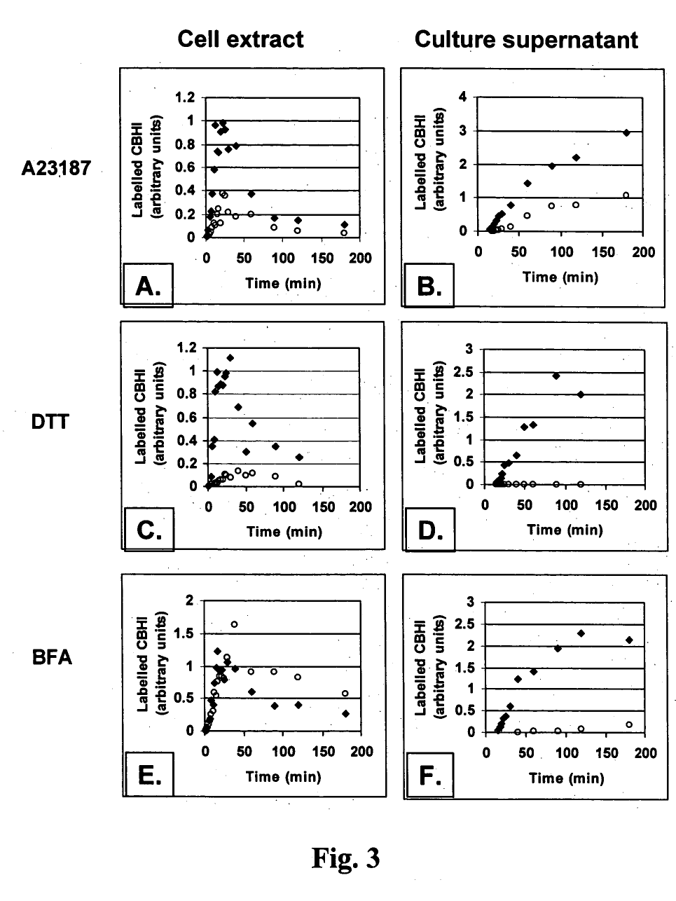

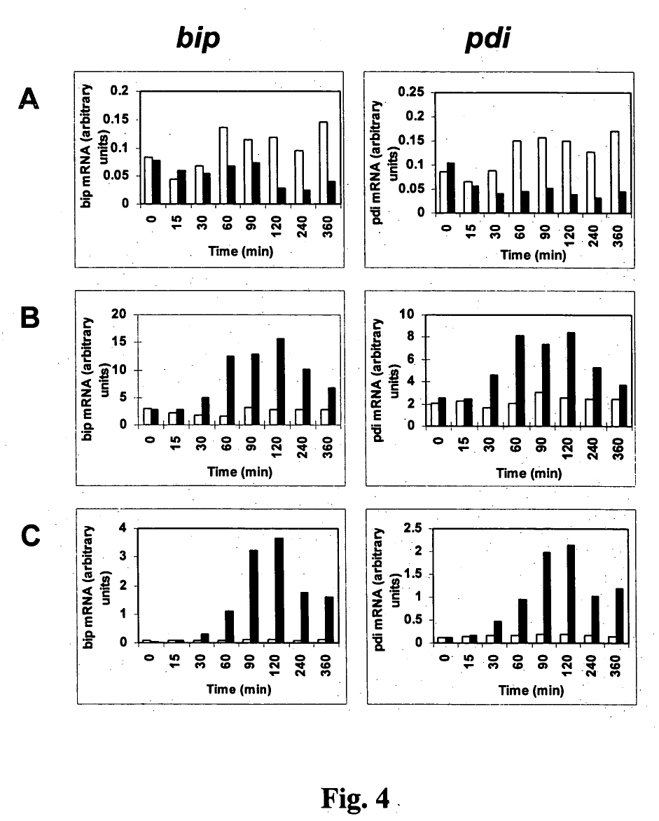

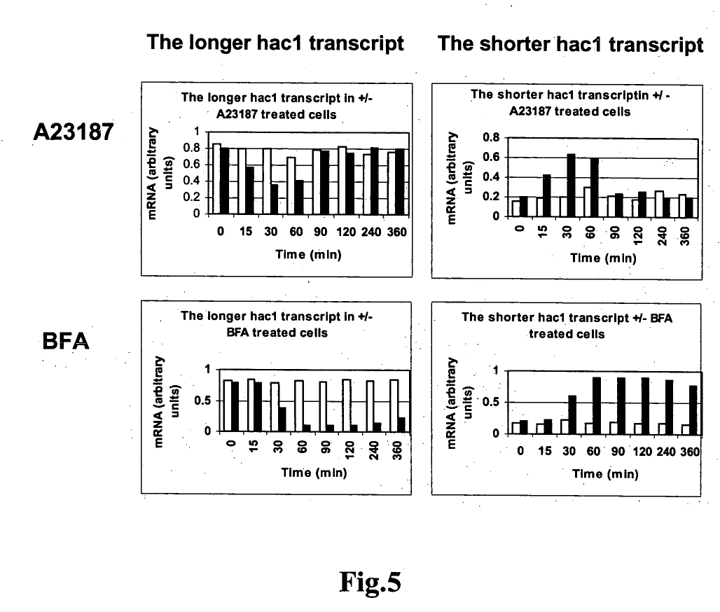

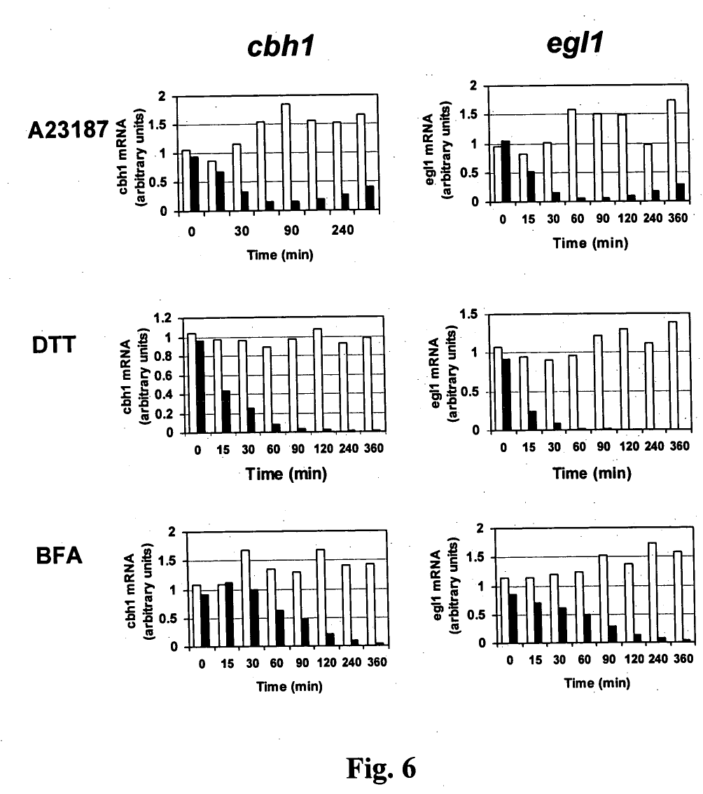

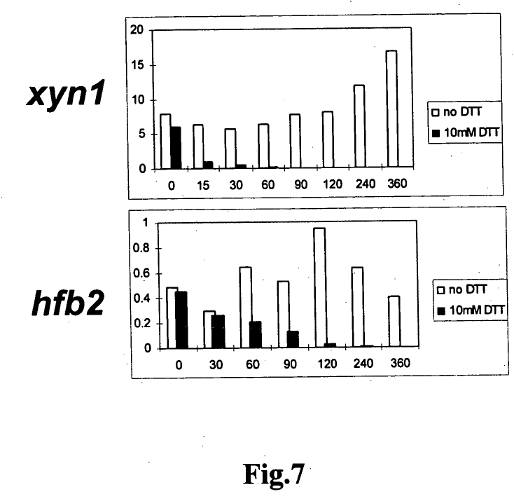

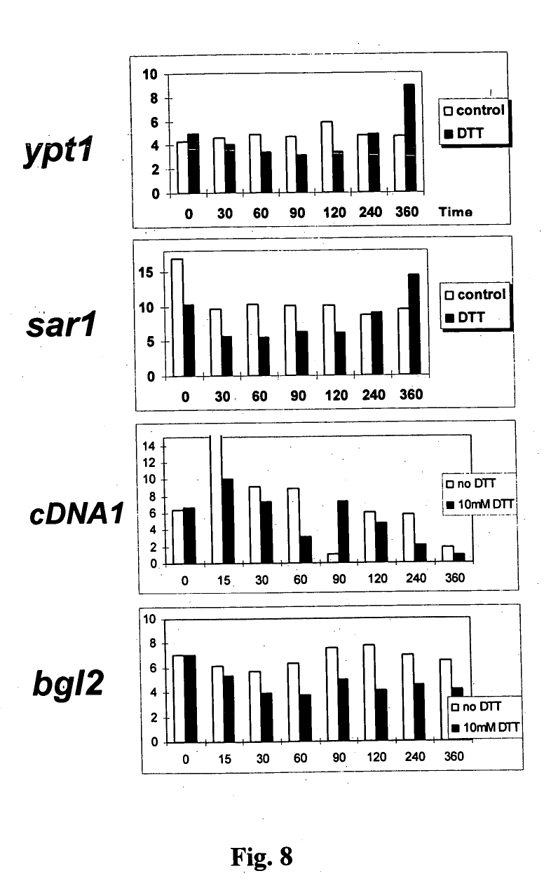

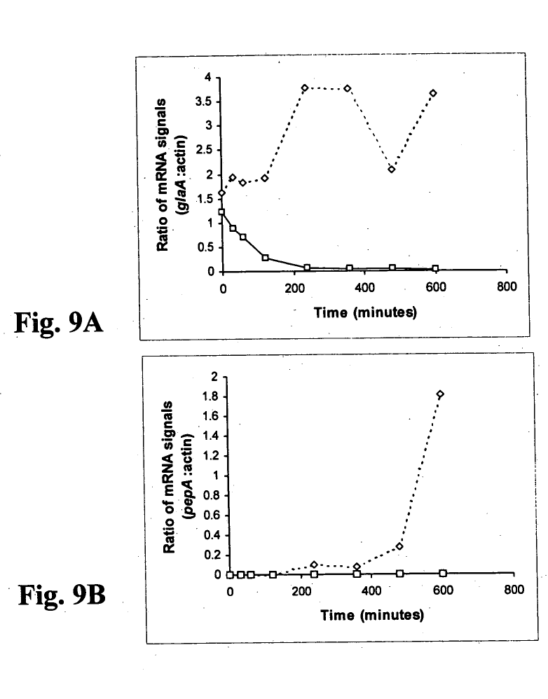

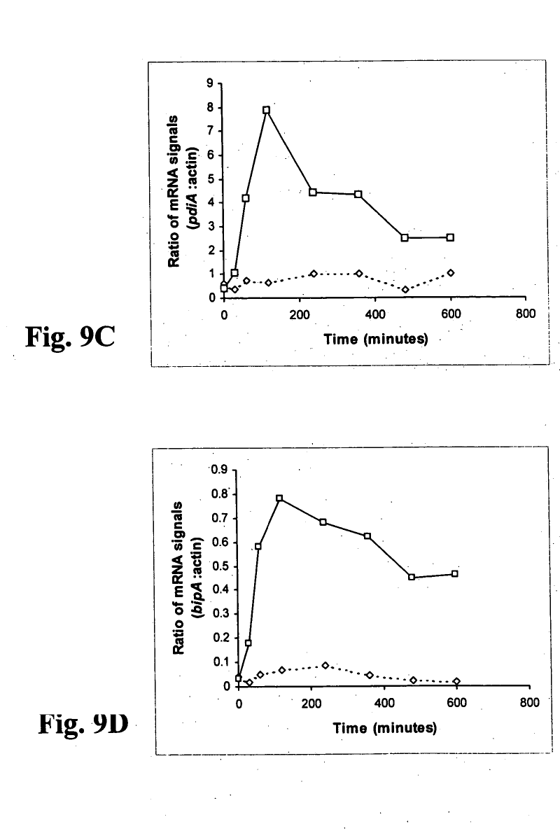

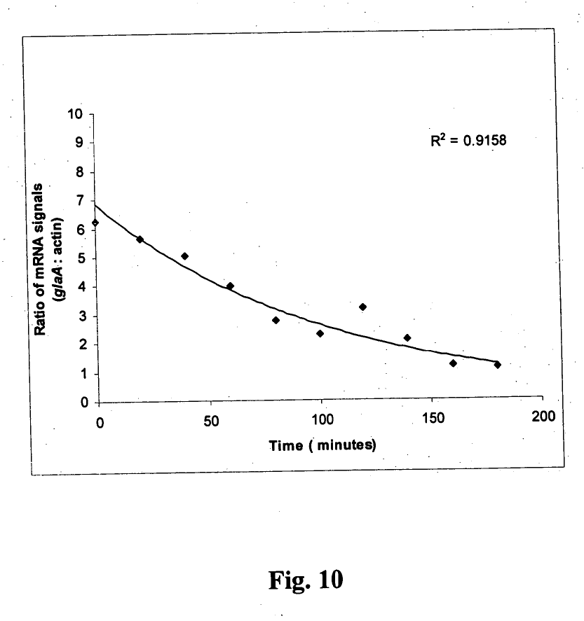

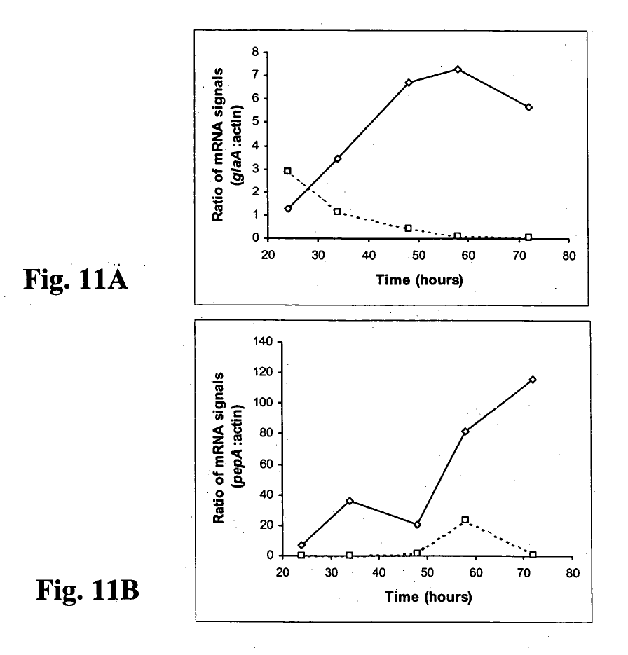

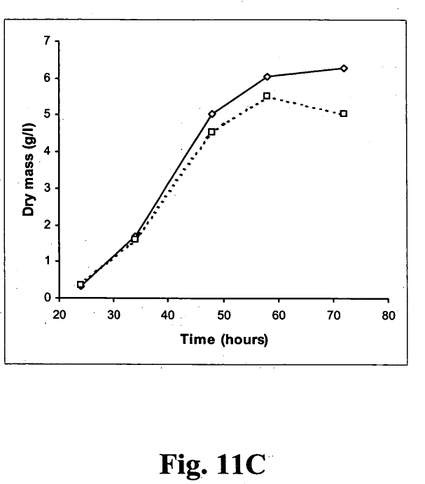

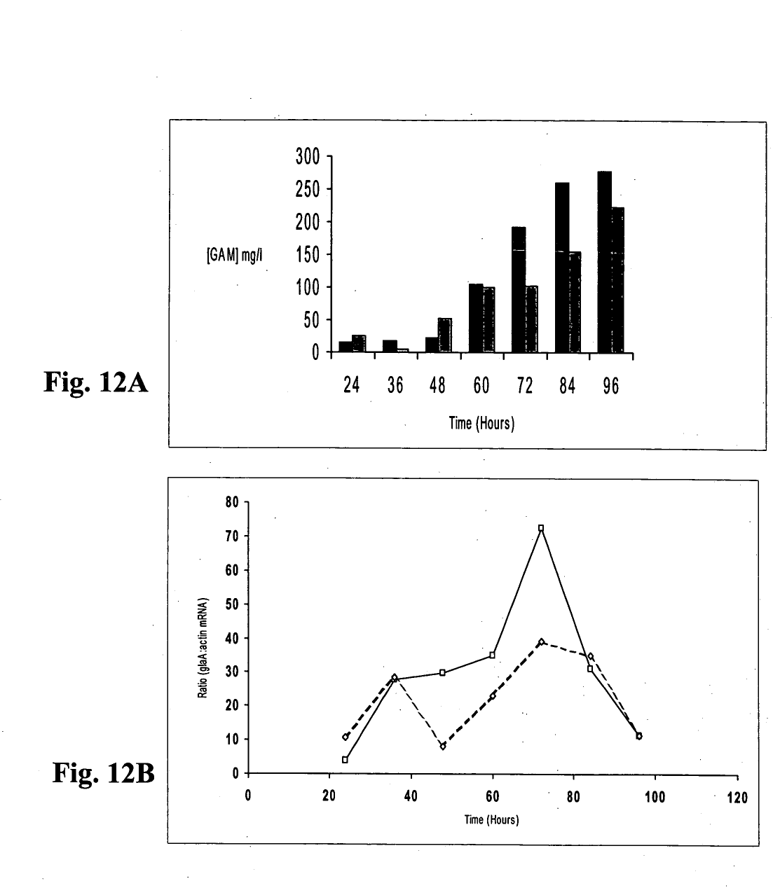

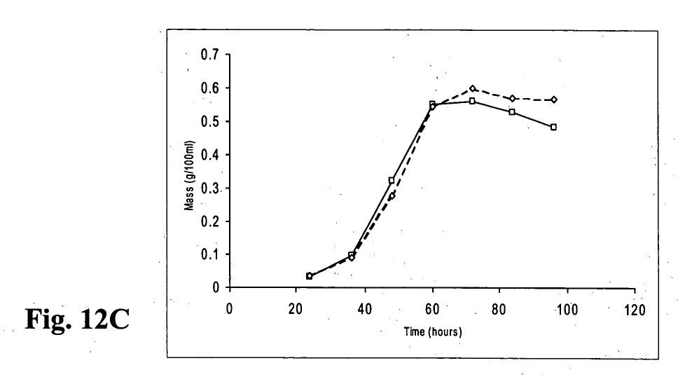

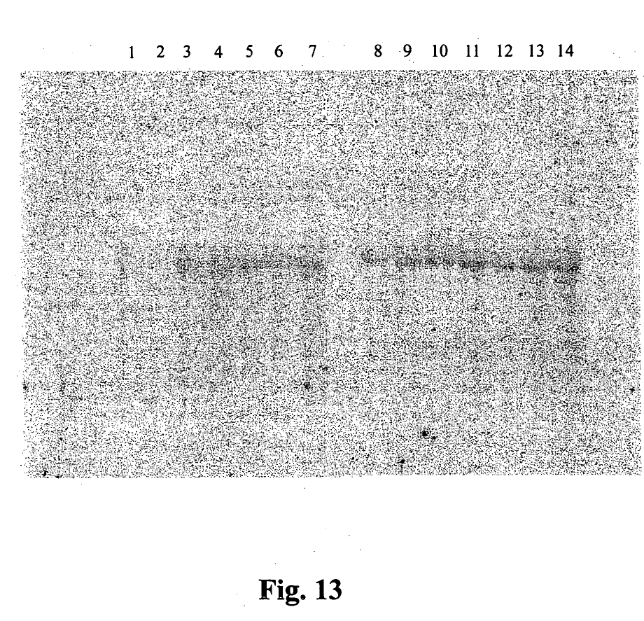

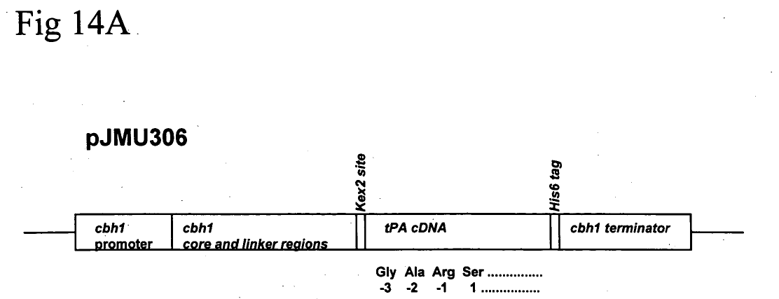

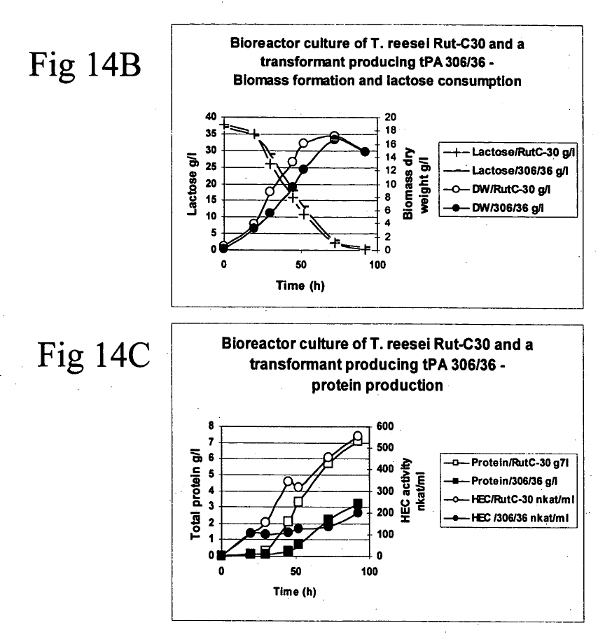

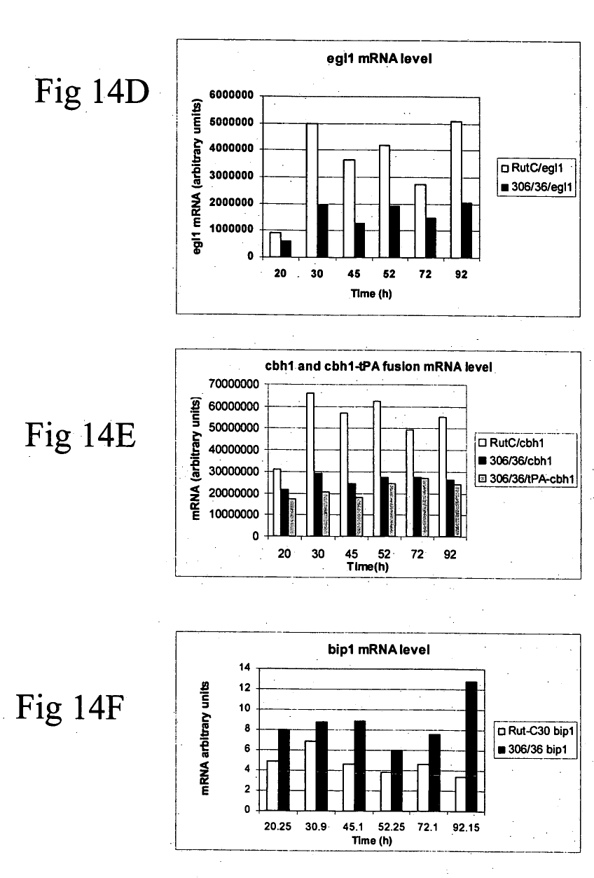

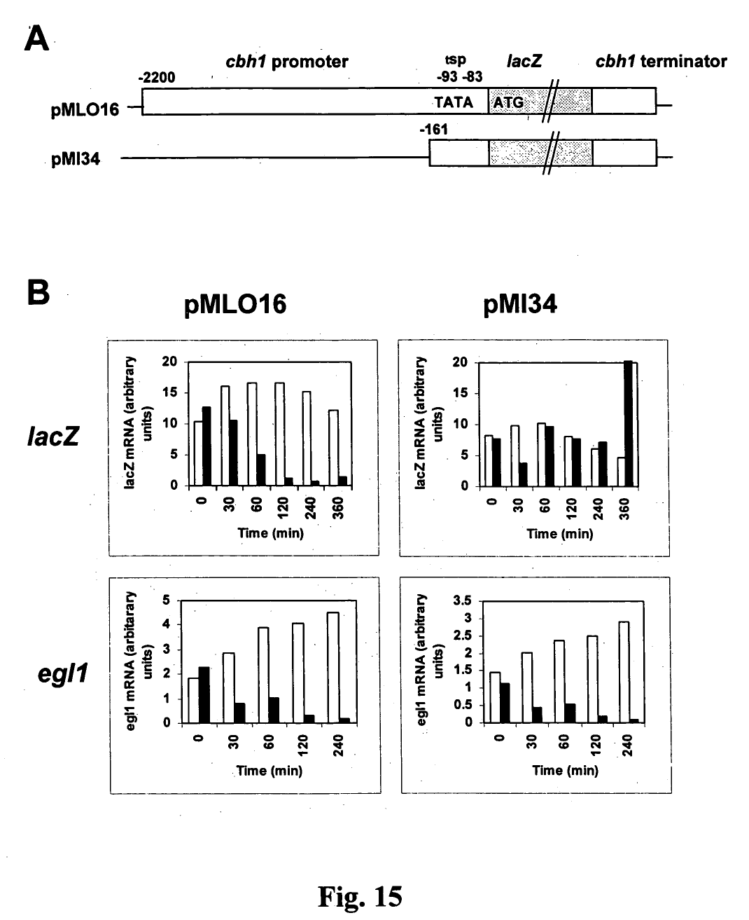

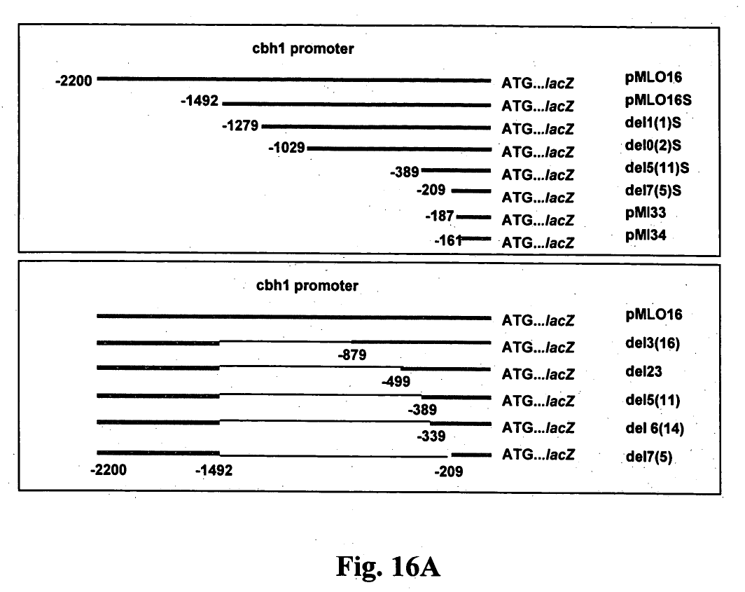

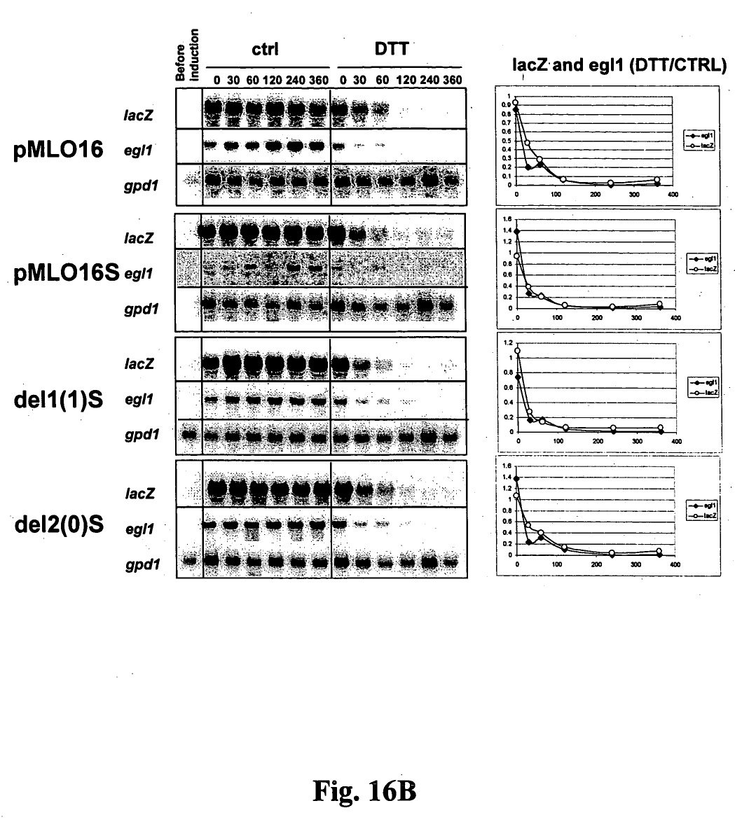

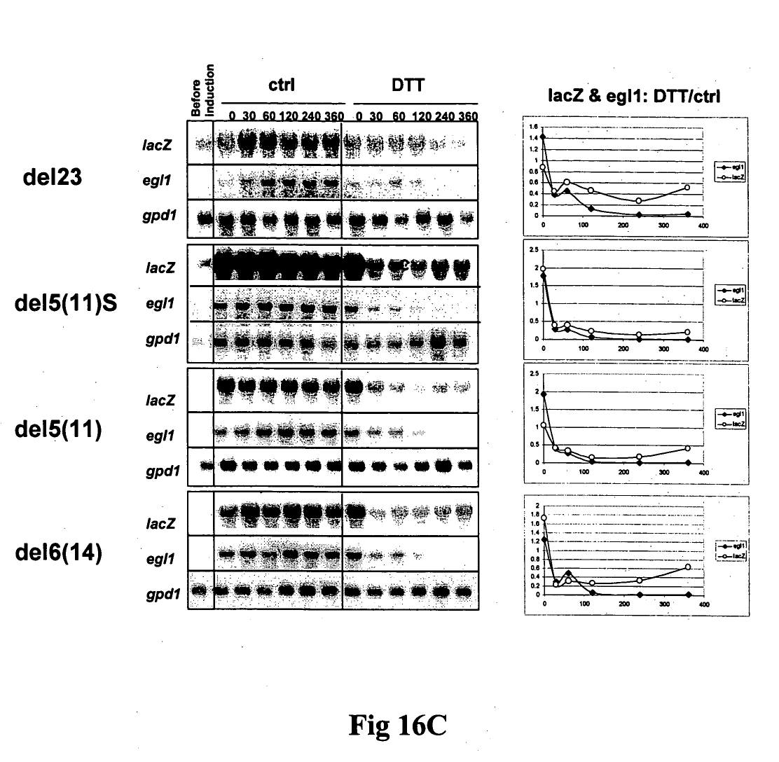

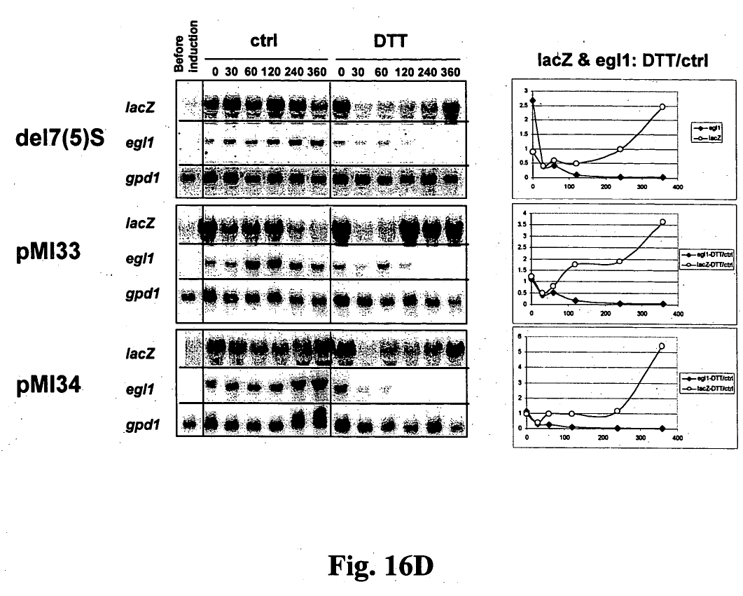

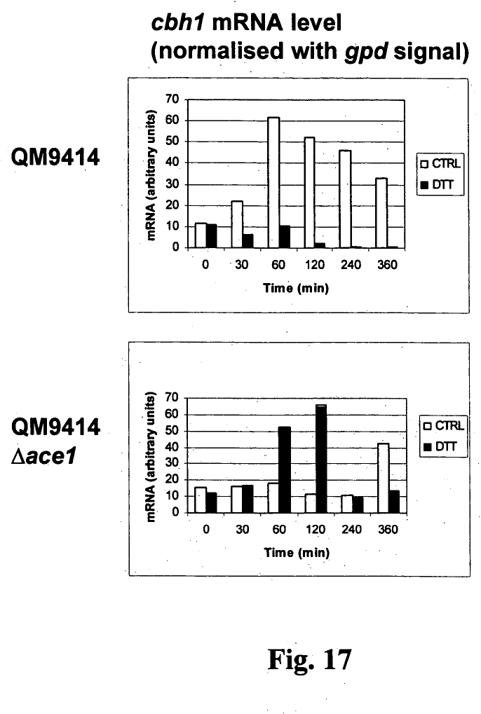

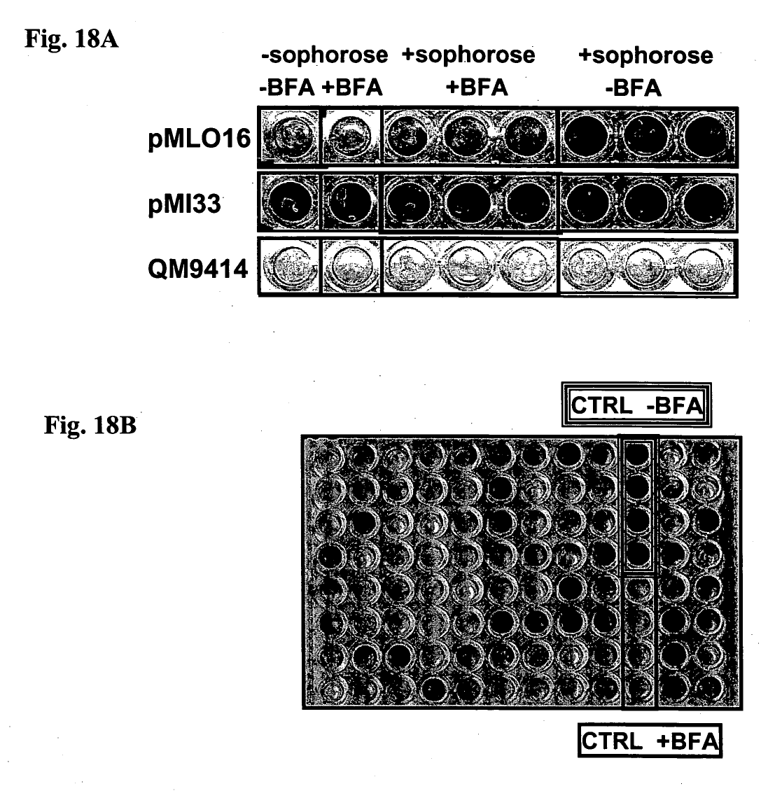

The rate of total protein synthesis and the rate of total protein secretion was measured as the amount of radioactivity incorporated into TCA insoluble material per time unit in cell extracts and in culture supernatant (FIG. 1., the radioactivity in the TCA insoluble material is shown per mg of biomass dry weight, and the time point 0 minutes corresponds to the addition of the labelled methionine). The rates were deduced from the values measured during the first 15-45 minutes of the treatment. In the presence of DTT or BFA the rate of total protein synthesis was not affected, whereas the treatment with the ionophore reduced the protein synthesis rate to 51% of that in the control cells. Production of extracellular labelled proteins was inhibited rather efficiently in cultures treated with DTT or BFA. In these cultures the secretion rate of total labelled proteins into the culture medium was only 5% of that in the control cells. In addition, in BFA treated cultures the production of extracellular proteins was markedly delayed compared to the control cultures. In cultures treated with the ionophore A23187, the rate of production of labelled proteins the culture medium was reduced to 23% of that in the non-treated cultures. The rates of protein synthesis and secretion are summarised in the Table 1 (in the Table 1 the values for the rates are shown as percentage of the values in the non-treated control cultures). The result indicates that DTT and BFA do not hinder protein synthesis, but block protein transport from the cells, whereas A23187 has also an inhibitory effect on protein synthesis.

The effect of the treatments on the synthesis of extracellular proteins, specifically, and on their transport was studied using the major cellulase produced by the fungus, cellobiohydrolase I (CBHI), as a model protein. The synthesis and secretion of the protein as well as changes in the pI pattern of the protein during the transport was monitored using 2D gel electrophoresis (as described in Pakula et al. 2000; FIG. 2 shows labelled CBHI at different time points of the labelling experiment analysed in 2D gels, pH range of approx. 3.5-4.5 from left to right in each panel). In cell extracts prepared from cultures treated either with DTT or BFA, only the very first nascent pI forms can be detected indicating that the protein is not fully posttranslationally modified in the biosynthetic pathway. In DTT treated cultures, no production of labelled CBHI into the culture medium was detected, and in BFA treated cultures only a minute amount of CBHI was secreted at the late stages of the labelling experiment (the production rate was 4% of that measured in the control cultures). The result suggests that in these conditions the transport of the protein is blocked before the protein reaches the compartment where the modifications causing the heterogeneity in the pI take place. However, the minute amount of CBHI detected in the culture medium of the BFA treated cultures had gained the full pI pattern indicating that a minor portion of the protein is modified and transported, but the amount of the fully processed forms of the protein is too low to be detected in the cell extracts. The effect of the treatment with the ionophore A23187 on protein transport was less pronounced. Formation of the full pI pattern of CBHI was delayed by 15-20 minutes compared to the control cells, and CBHI with full pattern of the pI forms was secreted into the culture medium but with a delay.

The labelled CBHI from samples of cell extract and culture supernatant at different time points of the labelling experiment was analysed in 2D gels and quantified using a phosphorimager (Molecular Dynamics). Parameters, such as the synthesis and secretion rate of CBHI (the amount of labelled protein produced per time unit) as well as the average synthesis time and the minimum secretion time of CBHI, were determined (for the method see Pakula et al. 2000). The quantification of the labelled CBHI during the labelling experiment is shown in FIG. 3, and the deduced parameters describing the synthesis and secretion of CBHI in these conditions are summarised in the Table 1. The average synthesis time of full-length CBHI was not affected in the DTT and BFA treated cultures, being in accordance with the result that total protein synthesis is not affected by these treatments (see above). The minimum secretion time of the molecule measured in the BFA treated cultures was increased from 11 minutes to 69 minutes, and in the DTT treated cultures the parameter could not be determined because of the very low amount of extracellular protein produced in these conditions. Treatment of the cultures with the ionophore A23187 had an effect on CBHI synthesis as well as on transport of the protein.

The minimum secretion time of CBHI was increased by 10 minutes in cultures treated with A23187 when compared to the control cultures, and the synthesis time of CBHI was 3-4 minutes longer than in the control cultures.

Surprisingly, although the treatment with DTT or BFA did not reduce the rate of total protein synthesis or prolong the time required for the synthesis of CBHI molecules, it was found out that the rate of CBHI synthesis (the amount of labelled CBHI synthesised per time unit) was reduced in cultures treated with DTT or BFA. (In the Table 1., the rates are shown as percentage of the values measured in the control cultures.). In the DTT treated cultures the CBHI synthesis rate was 4-24% of the one measured in control cultures and in the BFA treated cultures 52%. Most of the CBHI synthesised remains intracellular. The rate of CBHI production into the culture medium could not be measured in the DTT treated cultures, and in BFA treated cultures it was 4% of the one measured in the control cultures. In cultures treated with the ionophore A23187 the rate of CBHI synthesis was affected to greater extent than the total protein synthesis rate. The rate of CBHI synthesis was 26% of that measured in the control cells, and the total protein synthesis rate 51%. The protein secretion rate into the culture medium was reduced to the same extent as the synthesis rate of CBHI (27% of that measured in the control cultures).

The results show that the treatment with BFA or DTT clearly hindered protein transport in Trichoderma, probably preventing protein transport further from the ER, whereas the treatment with A23187 caused only a slight delay in protein transport. The total protein synthesis activity was not affected in cultures treated either with DTT or BFA, whereas the synthesis rate of the secreted model protein CBHI was reduced specifically concomitant to the impairment of protein transport. In cultures treated with A23187, a clear reduction in the total protein synthesis rate was measured, but the synthesis rate of CBHI was affected to greater extent compared to the effects on total protein synthesis.

TABLE 1
The effect of treatment with A23187, DTT or BFA
on protein synthesis and secretion in T. reesei.

| | Total protein | Total protein | CBHI | CBHI | Average time of | Minimum secretion |
|---|---|---|---|---|---|---|
| | synthesis rate | secretion rate | synthesis rate | secretion rate | CBHI synthesis | time of CBHI |
| Untreated cells | 100% | 100% | 100% | 100% | 5.7 min | 11 min |
| A23187 | 51% | 23% | 26% | 27% | 9 min | 21 min |
| BFA | 100% | 5% | 52% | 4% | 4.6 min | 69 min |
| DTT | 105% | 5% | 4-24% | n.d. | 4.3 min | n.d. |

(n.d. = not detected)

Transcript Levels of Genes Coding for the Foldase PDII, the Chaperon BIPI, and the Transcription Factor HACI Mediating the UPR Response in T. reesei Cultures Treated Either with the Ca²⁺-Ionophore A23187, DTT or BFA

Northern analysis of the samples collected during the treatment with A23187, DTT or BFA was carried out to study the effect of the treatments at transcriptional level. The hindrance in protein transport and folding in cultures treated with DTT or BFA was manifested also as activation of the unfolded response (UPR) pathway as indicated by the induction of pdi1 and bip1 genes (FIG. 4, the result has been reported earlier for pdi1; Saloheimo et al. 1999), as well as by the expression of the shortened, actively translated form of the hac1 transcript that mediate the UPR response (FIG. 5 shows the signals of the short and longer forms of the transcript normalised with the total hac1 signal at each time point). In the cultures treated with A23187 the protein transport was only slightly affected and the total amount of protein synthesised was diminished. In these conditions no induction of pdi1 and bip1 was observed (FIG. 4). However, a transient and rather weak expression of a short form of hac1 mRNA was observed also in A23187 treated cells indicating some effect also on the UPR pathway (FIG. 5).

Transcript Levels of Genes Coding for the Endogenous Secreted Proteins in T. reesei Cultures Treated Either with the Ca²⁺-Ionophore A23187, DTT or BFA

In cultures treated either with DTT or BFA, CBHI was synthesised with a reduced rate compared to non-treated control cells, whereas total protein synthesis was not affected in these conditions. In cultures treated with A23187, the synthesis of CBHI was retarded to a greater extent compared to the total protein synthesis in the treated cultures. Northern analysis of the samples prepared from the cultures treated with the drugs showed that the mRNA level of cbh1 decreased markedly during the treatment (FIG. 6, the cbh1 signals normalised with the signals of gpd at differnet time points of the treatment). The reduced mRNA level seem to explain, at least partly, the reduced synthesis rate of the protein in the labelling experiment. In DTT and A23187 treated cultures the reduction in the mRNA level of the gene occurred with kinetics corresponding to the measured half-life of the mRNA. In BFA treated cultures the decrease was somewhat slower. Similar reduction was observed in the egl1 mRNA level during the treatments (FIG. 6, signals normalised with the signals of gpd at different time points of the treatment). In addition, a Northern analysis of a broader set of genes was carried out from samples of DTT treated cultures. Transcript level of various other genes coding for extracellular proteins was reduced e.g. xyn1 and hfb2 (FIG. 7), indicating that many of the genes coding for extracellular proteins are under the feedback control in conditions where there are limitations in protein synthesis, folding or transport.

It is also evident that the down-regulation does not affect all the genes expressed by the fungus, but is common to a group of genes coding for extracellular proteins. In addition to the upregulated genes under the control of UPR, several examples of genes that were not down-regulated were found (FIG. 8). Interestingly, under these conditions the mRNA level of bgl2 coding for an intracellular β-glucosidase was not decreased, even though the gene is regulated in similar manner as cellulases in respect to the carbon source available for the fungus. The expression level of genes coding for proteins functioning in the vesicle transport, e.g. sar1 (Veldhuisen et al. 1997) and ypt1, were not affected by the treatment with DTT (Saloheimo et al. submitted). Other genes whose expression is not apparently affected by the DTT treatment are e.g. cDNA1 and gpd (glyceraldehyde-6-P-dehydrogenase). The gpd signal was used for normalisation the signals in the Northern analyses.

### Example 2

### Transcript Levels of Genes Coding for the Endogenous Secreted Proteins in Cultures of A. niger Treated with DTT

A. niger Strains, Cultivation Conditions and Methods Used for Sampling and Analysis of RNA

The Aspergillus niger strains used in the experiments were AB4.1 (van Hartingsveldt et al., 1987) and AS1.1 (Ngiam et al., 2000). Spores resuspended in 0.1% Tween 20 (Sigma, UK) were used to inoculate liquid cultures to a final density of 1×10⁵ spores per ml of medium. The strains were maintained on potato dextrose agar slopes (Difco, USA) with a supplement of 10 mM uridine for A. niger AB4.1. Slopes were grown at 30° C. until they had sporulated and made fresh for each experiment. ACMS/N/P medium (Archer et al., 1990) was used for all the experiments involving liquid culture. A. niger AB4.1 cultures were again supplemented with 10 mM uridine. Cultures were grown in 100 ml aliquots of medium in 250 ml conical flasks at 25° C. and 150 rpm. In the DTT stress experiments, AB4.1 cultures were grown for 44 hours before addition of 1 ml of 2M DTT solution to give a final concentration of 20 mM. Control AB4.1 cultures had an equivalent volume of water added. For the medium exchange experiment, cultures were grown for 44 hours at 25° C. and 150 rpm in ACMS/N/P. The mycelium was harvested through Miracloth (CalBiochem, USA) and washed with two 100 ml aliquots of medium with no carbon source that had been pre-warmed to 25° C. The mycelium was then transferred to pre-warmed flasks containing 100 ml ACMX/N/P with supplementation where appropriate and incubation was continued using the same conditions as before. ACMX/N/P differs from ACMS/N/P in containing 10 g xylose per litre instead of 10 g of soluble starch per litre.

Mycelia were harvested through two layers of Miracloth and flash frozen in liquid nitrogen. The mycelia were then ground under liquid nitrogen to a fine powder which was freeze dried in an Edwards Modulyo freeze drier for two days. Dry weights were established by weighing the mycelia after two days in the freeze drier and then drying for a further day. If no decrease in weight was observed over this period the culture was assumed to be completely dry.

Total RNA was extracted from 100 mg of freeze dried, ground mycelia using the RNeasy Plant Mini Kit (Qiagen, UK) according to the manufacturer's instructions. RNA was quantified by reading absorbances at 230, 260 and 280 nm on a Uvikon 850 spectrophotometer (Kontron Instruments, UK). Ratios of over 2.0 for the 260 nm:280 nm readings were accepted as being indicative of good quality RNA. RNA quality was also assessed by running samples on 7% formaldehyde gels (Sambrook et al., 1989). For northern blotting, 10 μg of RNA per lane was run on a 7% formaldehyde gel in MOPS running buffer (Sambrook et al., 1989) for 16 hours at 25V in a Life Technologies Horizon 11-14 submarine gel electrophoresis tank. Samples were prepared using Sigrna RNA loading dye (Cat.# R4268). After electrophoresis, the gel was washed in 5 changes of DEPC-treated water (Sambrook et al., 1989) for 20 minutes each wash and then soaked in 50 mM NaOH for 10 minutes. Transfer to Hybond XL nylon membrane (Amersham Intl., UK) was achieved using an Appligene vacuum blotter according to the manufacturer's instructions with 10×SSC (Sambrook et al., 1989) as transfer buffer. Transfer time was 2.5 hours. After transfer, the blot was soaked in 50 mM NaOH for 5 minutes and then rinsed in 2×SSC for 30 seconds before being allowed to air dry overnight.

Probes for the northern blots were labelled using the Megaprime labelling kit and α-³²P dATP (both Amersham Intl., UK) according to the manufacturer's instructions. The glaA probe was a 637 bp fragment corresponding to co-ordinates +1059 to +1696 in the sequence of the A. niger glucoamylase gene (Boel et al., 1984). The actin probe was a 765 bp fragment corresponding to co-ordinates +889 to +1654 in the γ-actin gene of A. nidulans (Fidel et al., 1988). The pdiA probe was a 303 bp fragment corresponding to co-ordinates +63 to +365 in the sequence of the pdiA gene of A. niger (Ngiam et al., 1997). The pepA probe was a 445 bp fragment corresponding to co-ordinates +1186 to +1631 in the A. awamori aspergillopepsin gene (Berka .et al., 1990). The bipA probe was a 445 bp fragment corresponding to co-ordinates +712 to +1156 of the A. niger bipA gene (van Gemeren et al., 1997) All of the probes were amplified by PCR from A. niger genomic DNA and purified from agarose-TAE gels using the Qiaquick gel extraction kit (Qiagen, UK).

Blots were pre-hybridised at 65° C. in Hyb9 hybridisation solution (Puregene, USA) for 30 minutes prior to the addition of the probe DNA. The hybridisation was then carried out overnight at 65° C. Blots were washed twice in 2×SSC, 0.1% SDS for 15 minutes at 65° C. and then once in 0.1×SSC, 0.1% SDS for 30 minutes at 65° C. Blots were visualised and the band intensities quantified using a FujiFilm BAS1500 phosphorimaging system. RNA loadings were normalised using the γ-actin probe. The figures shown in the graphs represent the ratio between the target mRNA signal and that of γ-actin. This is dependent on the time of exposure for the blots on each phosphorimage plate. Because the values on the different graphs do not represent absolute levels of the transcripts they are not directly comparable.

The Effect of DTT on Transcript Levels of Genes glaA, pepA, pdiA and bipA in A. niger Cultures

FIG. 9. shows the results from a DTT time course experiment running over 10 hours (from the addition of the stress agent, average signals of three determinations). Part (A) shows the effect on the steady state RNA levels for the glaA gene over this period. It can be seen clearly that in the DTT-treated cultures the amount of mRNA drops steadily over time, with a half-life of about 70 minutes. This correlates well with data from a medium exchange experiment carried out in this lab (FIG. 10.) which shows that the T½ of glaA mRNA is ca. 70 minutes in the absence of glaA mRNA synthesis. The result in FIG. 9A therefore suggests that DTT treatment inhibits the transcription of glaA and that the decline in the level of the glaA mRNA is due to its normal degradation within the organism. FIG. 9B shows the effect of DTT stress on another secreted protein, aspergillopepsin (pepA). This gene is only induced when the pH of the medium becomes more acidic and so transcription does not occur until late in the time course. The data show that, though there is an increase in the levels of pepA mRNA in the control cultures, there is no significant increase in the DTT treated cultures. FIGS. 9C and D show the effects of DTT on genes involved in the unfolded protein response. Both of the genes shown, pdiA and bipA, show a rapid response to the addition of the stress agent. This response does not appear to be transient but, conversely, is long lived. It is not known whether this is due to the production of messenger RNA for an extended period after addition of the DTT or due to long half lives for the mRNAs involved.

### Example 3

### The Transcript Levels of Genes glaA and pepA in Cultures of A. niger Expressing pdiA Antisense Transcript Under the Control of Glucoamylase Promoter

The expression of glaA and pepA has been compared in A. niger strain expressing pdiA antisense construct and in its parental strain. The methods for cultivation of the strains and RNA analysis have been described in the Example 2.

FIG. 11 show data obtained from a comparison of A. niger AS1.1, which contains multiple copies of a pdiA antisense sequence under the control of the glucoamylase promoter, to the parental strain A. niger AB4.1 when grown on medium containing starch as a carbon source. Panel (a) shows the effect on the mRNA levels for the glaA gene. It can be seen that from the first time-point at 24 hours the levels of glaA mRNA in the AS1.1 strain show a gradual decline while those for AB4.1 increase. From this and Panel (a) in FIG. 1 it can be seen that the levels of glaA mRNA in the parental strain (AB4.1) are actually increasing in relation to the level of γ-actin which is used for normalisation. This may be due in part to the long half-life of the glaA mRNA that would mean that the rate of breakdown of the mRNA is significantly slower than its rate of production giving rise to an ever-increasing population for this mRNA. In panel (b) the effects on the transcription of the pepA gene are shown. Again there are significantly lower levels of mRNA in the AS1.1 strain than in the parent, AB4.1. Panel (c) shows the dry weight determinations for the experiments, which show that there is no significant effect on the growth of the fungus when the antisense construct is expressed.

### Example 4

### The Transcript Levels of the Gene glaA and the Levels of Secreted Glucoamylase in Cultures of A. niger Constitutively Expressing pdiA Antisense Transcript Under the Control of the gpdA Promoter

The expression of glaA has been compared in an A. niger strain constitutively expressing pdiA antisense cDNA under the control of the gpdA promoter (strain ASG67) and in its parental strain. The methods for cultivation of the strains and RNA analysis have been described in Example 2. For analysis of secreted glucoamylase protein levels, a 7 ml sample of culture filtrate from each flask was collected and stored at −20° C. until required. The method used for determination of glucoamylase was that of MacKenzie et al., 1994.

FIG. 12 shows data obtained from a comparison of A. niger ASG67, which contains multiple copies of a pdiA antisense sequence under the control of the glyceraldehyde-3-phosphate dehydrogenase promoter, to the parental strain AB4.1 grown on medium containing starch as a carbon source. Panel (a) shows the effect on the levels of secreted glucoamylase. It can be seen that, although the levels of secreted glucoamylase increase in both strains over time, the levels for the antisense strain are lower than those for the parental strain (AB4.1), especially later in the growth of the fungus. In panel (b) the effects on the transcript levels for the glaA gene can be seen. After initially reaching the same transcript level at 36 hours, there is a gradual increase in transcript levels in the parental strain (AB4.1) which is not mirrored in the pdiA antisense strain (ASG67). Panel (c) shows the dry weight determinations for the experiments which demonstrate that there is no significant effect on the growth of the fungus when the antisense construct is expressed.

### Example 5

### The Splicing of the hacA Transcript in A. niger Constitutively Expressing pdiA Antisense Transcript

The splicing of the hacA transcript, which encodes the positively acting regulatory factor for the unfolded protein response, has been analysed in an A. niger strain which constitutively expresses a pdiA antisense sequence and in its parental strain. The methods for cultivation of the strains and RNA analysis have been described in Example 2. The hacA probe used in the experiment was the hacA cDNA isolated at VTT. The same cultivations were used to provide the data in Example 4.

FIG. 13 shows a northern blot for hacA over time. If there was induction of the unfolded protein response (UPR) there would be evidence for a second mRNA species slightly lower on the gel than the species which is present. The mRNA present is of the correct size for unspliced hacA. These data suggest that there is no induction of the UPR which implies that the transcriptional down-regulation mechanism is distinct from the UPR and is controlled in a different manner.

### Example 6

### The Expression Level of Genes Coding for Endogenous Secreted Proteins in T. reesei Strains Producing Heterologous Proteins

Strains, Cultivation Conditions and Methods Used in the Analysis of the Cultures.

T.reesei Rut-C30 strain producing human tissue plasminogen activator (tPA, Verheijen et al. 1986) was constructed by transforming the parental strain with the expression cassette shown in FIG. 14A using the methods described in Penttilä et al. 1987.

The tPA producing strain and the parental strain Rut-C30 were cultivated in bioreactors in parallel. The culture medium used was lactose-based buffered medium used at VTT Biotechnology (lactose 40 g/l, peptone 4 g/l, yeast extract 1 g/l, KH₂PO₄ 4 g/l, (NH₄)₂SO₄ 2.8 g/l, MgSO₄×7H₂O 0.6 g/l, CaCl₂×2H₂O 0.8 g/l, supplemented with trace elements). Dry weight of the biomass was measured as described in Example 7. Lactose concentration in the culture medium was determined using a kit obtained from Boehringer Mannheim, total protein in the culture medium was measured using the Protein Assay obtained from BioRad, HEC activity was measured as described (in Bailey and Nevalainen, 1981; IUPAC, 1987) and the tPA concentration was measured using the EIA kit provided by TNO (the Netherlands). RNA isolation and Northern analysis was performed as described in the Examples 1, 7, 8, and 9.

Expression of the Endogenous Extracellular Proteins in a tPA Producing Strain and its Parental Strain

Production of endogenous secreted proteins and the expression of the corresponding genes was studied in T. reesei Rut-C30 and in a transformant producing a tPA (human tissue plasminogen activator), which is an example of a heterologous protein that is very poorly produced by the fungus, and expected to induce various stress responses in its host. The transformant has been estimated to harbour approximately five copies of the expression cassette, from which tPA is produced as a CBHI-fusion protein under cbh1 promoter.

To compare protein production and expression of the corresponding genes in the two strains, parallel cultivations in bioreactors were carried out. Formation of biomass and consumption of the carbon source, lactose, was measured during the cultivation to monitor growth (FIG. 14B). Total protein and cellulase activity (activity against the substrate HEC, measuring mainly endoglucanase activity) produced into the culture medium were measured throughout the cultivation (FIG. 14C). Northern analysis was carried out to analyse egl1 (FIG. 14D), cbh1 (FIG. 14E) and bip1 (FIG. 14F) expression in the cultures. The signal of actin was used for normalisation of the signals in the Northerns.

Even though, the two strains grew rather similarly during the cultivation, it was obvious that the tPA producing strain produced much less total protein and cellulase activity into the culture medium compared to the parental strain. The tPA produced by the transformant only a minor proportion of the total protein produced, the highest yield obtained is 25 mg/l. In accordance with the low protein production in the tPA producing culture, the expression levels of egl1, coding for the extracellular endoglucanase I, and cbh1, coding for cellobiohydrolase I, were lower in the culture producing tPA. Expression of the chaperon gene bip1 was induced in the tPA producing culture indicating activation of stress responses, such as UPR, by production of the heterologous protein. Thus the low expression levels of endogenous genes coding for secreted proteins in the transformant could be due to the down-regulation mechanism active during secretion stress.

### Example 7

### Expression of the Reporter Gene lacZ Under Full-Length cbh1 Promoter and a Shortened Minimal cbh1 Promoter in DTT Treated Cultures of T. reesei—the Role of the Promoter Sequence in the Down-Regulation

Cultivation Conditions, and Methods Used for Analysis of the RNA Samples

The strain QM9414 (Mandels et al. 1971) and its derivatives pMI34 and pMLO16 expressing Escherichia coli lacZ under cbh1 promoter (Ilmén et al 1996) were cultivated on the minimal medium containing 0.05% proteose peptone and 20 g/l sorbitol or glycerol. 8×10⁷ spores were inoculated per 200 ml of growth medium and the cultures were grown in conical flasks at 28° C. with shaking at 210 rpm. α-Sophorose (1 mM) was added after 23 h and after 32 h of cultivation to induce cellulase gene expression on sorbitol medium. Treatment of the cultures with 10 mM DTT was started after 40 h of cultivation. Mycelial samples for RNA isolation were collected and subjected to Northern analysis as described in the Example 1. Dry weight of the cultures was measured before and after the sophorose induction and the treatment with DTT by filtering and drying mycelium samples at 105° C. to constant weight (24 h). The dry weight in the cultures was 1.1-1.4 g/l at the beginning of the treatment with DTT.

The Reporter Gene Activity Under cbh1 Promoter During DTT Treatment

To study whether the feedback regulation of the mRNA level was mediated by the promoter sequence of the gene involved a reporter gene system was used. A schematic view of the reporter gene expression cassettes is shown in FIG. 15A. The E. coli lacZ gene was expressed under a cbh1 promoter in the strain T. reesei, either under a full-length cbh1 promoter of 2.2 kb or under a minimal promoter of 161 bp, and the expression levels were studied during DTT treatment of the strains. The quantification of the lacZ signal normalised with the signal of gpd1 is shown in FIG. 15B. The lacZ transcript level is down-regulated during DTT treatment only when expressed under the full-length cbh1 promoter. However, no down-regulation was observed if a minimal cbh1 promoter containing the putative TATA-box and the transcription start sites was used for lacZ expression, even though the short promoter is functional and even inducible with sophorose. The transcript level of egl1 was analysed in both of these strains to control that the down-regulation mechanism is functional in these strains under these conditions. The result indicates that sequence elements in the cbh1 promoter are required for the down-regulation, and a mechanism other than the instability of the mRNA is involved in the process.

### Example 8

### Expression of the Reporter Gene lacZ Under the Control of Shortened cbh1 Promoter in DTT Treated Cultures of T. reesei—A Method for Identification of Promoter Regions Mediating the Down-Regulation of the Promoter under Secretion Stress Cconditions

T. reesei strains harbouring E.coli lacZ gene under shortened cbh1 promoters were cultivated and treated with DTT as described in the Example 7, and the expression of the lacZ gene was analysed (as in the example 7). FIG. 16A shows the schematic presentation of the cbh1 promoter constructs used for lacZ expression in the different strains. The Northern analysis of lacZ, egl1 and gpd1 mRNA level in the cultures treated with DTT and in the non-treated cultures is shown in the FIGS. 16B, C and D. The mRNA level of egl1 was analysed as an example of an endogenous gene subjected to the down-regulation under secretion stress conditions (e.g. in DTT treated cultures), and the signal for gpd1 was used as a control for loading of the samples. The signals of lacZ and egl1 mRNA were quantified and normalised with the signal of gpd1, and the ratio of the signal in the DTT treated sample to the signal in the control samples at different time points of the treatment is shown as graphs. In the strains harbouring the constructs with cbh1 promoters of 1029 bp in length or longer (shown in FIG. 16B), the expression of lacZ was decreased during the treatment with DTT to a similar extent as in the strains expressing the gene under the full-length cbh1 promoter of 2.2 kb. In strains expressing the lacZ gene under cbh1 promoters of 339 bp to 499 bp in length (shown in FIG. 16C), the level of lacZ mRNA was clearly decreased during the treatment with DTT, but not to the same extent as if expressed under the full-length cbh1 promoter, and not to the same extent as the mRNA level of egl1 that was used as an internal control for down-regulation in the strain. In strains expressing the lacZ gene under the shortened promoters of 161 bp to 209 bp in length (shown in FIG. 16D), a strong expression of lacZ (as compared to the signal in the non-treated cultures) was detected during the treatment with DTT. The results indicate that in the case of the cbh1 promoter, the regions involved in the decrease in the expression level during the DTT treatment are located within the 1029 bp region upstream of the translation start codon, the most important regions being located in the regions 500-1029 bp and 209-339 bp upstream of the start codon.

### Example 9

### Expression of cbh1 in DTT Treated Cultures of T. reesei QM9414 and its Derivative Harbouring a Deletion in the Gene ace1

To study the possible role of the cellulase regulator ace1 in the down-regulation of the cellulase promoters under the secretion stress conditions, cultures of T. reesei QM9414 and a derivative of the strain with a deletion in the gene ace1 (Saloheimo et al. 2000) were treated with DTT and analysed for cellulase expression. The strains were cultivated on sorbitol containing medium, induced with sophorose, and treated with 10 mM DTT as described in the Example 7. Sampling of the mycelium for RNA analysis as well as the Northern analyses have been described in the Examples 1 and 7 as well. The transcript level of cbh1 was quantified during the treatment and the signals were normalised with the ones of gpd1 (FIG. 17).

The cbh1 is subjected to down-regulation during DTT treatment in cultures of QM9414 in a similar manner as has been shown for the strain T. reesei Rut-C30 (Example: 1). However, in cultures of the QM9414 strain harbouring a deletion in ace1 grown on sorbitol. containing medium, the cbh1 is constitutively expressed also during treatment with DTT. In these specific conditions the ace1 activity seem to be required for the down-regulation of the cbh1 promoter. However, we have also evidence that in other culture conditions (e.g. on glycerol containing medium), the ace1 activity is not required, indicated that other factors, not yet known, are involved in this regulation mechanism.

### Example 10

### Isolation of Fungal Mutant Strains Defective in the Mechanism of Transcriptional Down-Regulation of Genes Under Secretion Stress Conditions

T. reesei strain pMLO16 expressing the E. coli lacZ reporter gene under the full-length cbh1 promoter was mutagenised using UV irradiation, and mutants capable of expressing lacZ under secretion stress conditions, in the presence of BFA, were screened for based on color reaction.

A spore suspension containing 10⁷ spores/ml was subjected to UV radiation leading to 15-46% viability of the spores. The mutagenised spores were cultivated on minimal medium containing sorbitol as a carbon source (as in the Example 7, except that pH 7.0 was used in this case) on microtiter plates, approx. 3 spores per well. After cultivation of 7 days, sophorose and brefeldin A were added to induce lacZ expression and and to generate secretion stress conditions at the same time. Induction of LacZ production in the presence of BFA was detected by the color reaction caused by addition of X-gal in the cultures. The lacZ expressing cultures were purified on PD plates, and the ability of the mutants for induction of the cbh1 promoter (controlling lacZ expression) in the presence of BFA was confirmed. The FIG. 18A. shows the lacZ activity in the control cultures of pMLO16 expressing lacZ under the down-regulatable full-length cbh1 promoter, in the strain pMI33 expressing the lacZ under a minimal promoter of cbh1 (not down-regulated in the secretion conditions, see also example 8), and in the lacZ negative strain QM9414. After sophorose addition, there is no lacZ production in the presence of BFA, whereas in the absence of BFA, lacZ is produced, as indicated by the color reaction. The FIG. 18B. shows an example of screening of the mutants in the microtiter plate cultures. The mutants expressing lacZ under the secretion stress conditions can be isolated based on the color reaction. As a control, the unmutagenised spores of pMLO16 were cultivated on the plates in the presence and absence of BFA (see the boxed wells; positive color reaction indicating lacZ production in the absence of BFA, and lack of color reaction in the presence of BFA)

References

Alberini, C. M., Bet, P., Milstein, C. & Sitia, R. (1990). Secretion of immunoglobulin M assembly intermediates in the presence of reducing agents. Nature 347, 485-487.

Archer, D. B, Jeenes, D. J, MacKenzie, D. A., Brightwell, G., Lambert, N., Lowe, G., Radford, S. E. and Dobson, C. M. 1990. Hen egg white lysozyme expressed in, and secreted from, Aspergillus niger is correctly processed and folded. Bio/Technology 8: 741-745.

Archer, D. B. & Peberdy, J. F. (1997). The molecular biology of secreted enzyme production by fungi. Cri. Rev Biotechnol 17, 273-306

Bailey, M. J. and Nevalainen, K. M. H. (1981) Induction, isolation and testing of stable Trichoderma reesei mutants with improved production of solubilizing cellulase. Enzyme Microb. Technol. 3, 153-157.

Braakman, I, Helenius, J. & Helenius, A. (1992). Manipulating disulphide bond formation and protein folding in the endoplasmic reticulum,. EMBO J 11, 1717-1722.

Boel, E., Hansen, M. T., Hjort, I., Hoegh, I. and Fiil, N. P. 1984. Two different types of intervening sequences in the glucoamylase gene from Aspergillus niger. EMBO Journal. 3: 1581-1585.

Broström, C. O., Chin, K. V., Wong, W. L., Cade, C. and Broström, M. A. (1989) Inhibition of translational initiation in eucaryotic cells by calcium ionophore. J. Biol. Chem. 264, 1644-1649.

Fidel, S., Doonan, J. H. and Morris, N. R. 1988. Aspergillus nidulans contains a single actin gene that has unique intron locations and encodes gamma actin. Gene 70: 283-293.

van Gemeren, I. A., Punt, P. J., Drint-Kuyvenhoven, A., Broekhuijsen, M. P., van't Hoog, A., Beijersbergen, A., Verrips, C. T. and van den Hondel, C. A. M. J. J. 1997. The ER chaperone encoding bipA of black Aspergilli is induced. by heat shock and unfolded proteins. Gene 198: 43-52.

Gouka, R. J., Punt, P. J. & van den Hondel, C. A. M. J. J. (1997). Efficient production of secreted proteins by Aspergillus: progress, limitations and prospects. Appl Microbiol Biotechnol 47, 1-11.

Harding, H. P., Zhang, Y. H. and Ron, D. 1999. Protein translation and folding are coupled by an endoplasmic reticulum-resident kinase. Nature 397: 271-274.

van Hartingsveldt, W., Mattem, I. E., van Zeijl, C. M., Pouwels, P. H. and van den Hondel, C. A. M. J. J. 1987. Development of a homologous transformation system for Aspergillus niger based on the pyrG gene. Molecular and General Genetics 206: 71-75.

Ilmén, M., Onnela, M.-L., Klemsdal, S., Keranen, S. & Penttilä, M. (1996). Functional analysis of the cellobiohydrolase I promoter of the filamentous fungus Trichoderma reesei. Mol Gen Genet 253, 303-314.

IUPAC (International Union of Pure and Applied Chemistry) (1987) Measurement of cellulase activities. Pure and Appl. Chem. 59, 257-268.

Jämsä, E., Simonen, M. & Makarow, M. (1994). Selective retention of secretory proteins in the yeast endoplasmic reticulum by treatment of cells with a reducing agent. Yeast 10, 355-370.

Mori, K. 2000. Tripartite management of unfolded proteins in the endoplasmic reticulum. Cell 101:451-454.

Lodish, H. F. and Kong, N. (1990). Perturbation of cellular calcium blocks exit of secretory proteins from the rough endoplasmic reticulum. J. Biol. Chem. 265, 10893-10899.

Lodish, H. F., Kong, N., and Wikström, L. (1992) Calcium is required for folding of newly made subunits of the asialoglycoprotein receptor within the endoplasmic reticulum. J. Biol. Chem. 267, 12753-12760.

MacKenzie, D. A., Jeenes, D. J., Belshaw, N. J., and Archer, D. B. (1993) Regulation of secreted protein production by filamentous fungi: recent development and perspectives. J. Gen. Microbiol. 139, 2295-2307.

MacKenzie, D. A., Gendron, L. C. G., Jeenes, D. J., and Archer, D. B. (1994) Physiological optimisation of secreted protein production by Aspergillus niger. Enzyme Microb. Technol. 16, 276-279.

Mandels, M., Weber, J., and Parizek, R. (1971) Enhanced cellulase production by a mutant of Trichoderma viride. Appl. Microbiol. 21, 152-154.

Margolles-Clark, E., Hayes, C. K., Harman, G. And Penttilä, M. (1996) Improved production of Trichoderma harzianum endochitinase by expression in Trichoderma reesei. Appl. Env. Microbiol. 62, 2145-2151.

Margolles-Clark, E., Ilmén, M. and Penttilä, M. (1997) Expression patterns of ten hemicellulase genes of the filamentous fungus Trichoderma reesei on various carbon sources. J Biotechnol. 57, 167-179.

Montenecourt, B. S. & Eveleigh, D. E. (1979). Selective screening methods for the isolation of high yielding cellulase mutants of Trichoderma reesei. Adv Chem Ser 181, 289-301.

Ngiam, C., Jeenes, D. J. and Archer, D. B. 1997. Isolation and characterisation of a gene encoding protein disulphide isomerase, pdiA, from Aspergillus niger. Current Genetics 31: 133-138.

Ngiam, C., Jeenes, D. J., Punt, P. J., van den Hondel, C. A. M. J. J. and Archer, D. B. 2000. Characterisation of a foldase, PDIA, in the secretory pathway of Aspergillus niger. Applied and Environmental Microbiology 66: 775-782.

Pakula, T. M., Uusitalo, J., Saloheimo, M., Salonen, K., Aarts, J., and Penttilä, M. (2000) Monitoring the kinetics of glycoprotein synthesis and secretion in the filamentous fungus Trichoderma reesei: cellobiohydrolase I (CBHI) as a model protein. Microbiology 146, 223-232.

Pelham, H. R. B. (1991) Multiple targets for Brefeldin A. Cell 67, 449-451.

Penttilä, M., Nevalainen, H., Rättö, M., Salminen, E., and Knowles, J. K. C. (1987) Gene 61, 155-164.

Penttilä, M. (1998). Heterologous protein production in Trichoderma. In Trichoderma & Gliocladium, pp. 365-382. Edited by G. E. Harman & C. P. Kubicek. London: Taylor & Francis LTD.

Saloheimo; A., Aro, N., Ilmén, M. and Penttilä, M. (2000) Isolation of the ace1 gene encoding a Cys2-His2 transcription factor involved in regulation of activity of the cellulase promoter cbh1 of Trichoderma reesei. J. Biol. Chem. 275, 5817-5825.

Saloheimo, M., Lund, M. & Penttilä, M. (1999) The protein disulphide isomerase gene of the fungus Trichoderma reesei is induced by endoplasmic reticulum stress and regulated by the carbon source. Mol Gen Genet 262, 35-45.

Sambrook, J., Fritsch, E. F. and Maniatis, T. 1989. Molecular Cloning: A Laboratory Manual. 2^{nd} Edition. Cold Spring Harbor Laboratory Press (New York).

Shah, N. and Klausner, R. D. (1993) Brefeldin A reversibly inhibits secretion in Saccharomyces cerevisiae. J. Biol. Chem. 268, 5345-5348.

Shamu, C. E., Cox, J. S. and Walter, P. 1994. The unfolded protein response pathway in yeast. Trends in Cell Biology 4: 56-60.

Veldhuisen, G. Saloheimo, M., Fiers, M. A., Punt, P. J., Contreras, R., Penttilä, M. & van den Hondel, C. A. M. J. J. (1997). Isolation and analysis of functional homologues of the secretion-related SARI gene of Saccharomyces cerevisiae from Aspergillus niger and Trichoderma. Mol Gen Genet 256, 446-455.

Verheijen, J. H., Caspers, M. P. M., Chang, G. T. G., de Munk, G. A. W., Pouwels, P. H., and Enger-Valk, B. E. (1986) Involvement of finger domain and kringle 2 domain of tissue-type plasminogen activator in fibrin binding and stimulation of activity by fibrin. EMBO J. 5, 3525-3530.

1. A method for producing a promoter for protein production in a fungal host, characterized in that the method comprises the steps of:
selecting a promoter of a secretable protein,
genetically modifying the promoter,
operable linking the promoter to the coding region of a reporter protein,
expressing the selected reporter protein under the regulation of the modified promoter in a fungal host under suitable culture conditions in secretion stress,
screening or selecting for cells showing enhanced or decreased protein expression of the selected reporter protein compared with the expression obtained with the non-modified promoter under the same conditions; and
recovering the fungal host comprising the promoter having modification in its transcriptional down-regulation mechanism.

2. The method according to claim 1, characterized in that the coding region of the selected reporter protein is the coding region of a secretable protein.

3. A method for producing a fungal host for protein production, characterized in that the method comprises the steps of:
selecting a promoter modified as in claims 1 or 2,
operable linking the modified promoter to the coding region of a gene encoding a selected secretable protein,
expressing the selected secretable protein under the regulation of the modified promoter in a fungal host under suitable culture conditions in secretion stress,
screening or selecting for cells showing enhanced or decreased protein expression of the selected secretable protein compared to the expression of secretable proteins under a non-modified promoter under the same conditions; and
recovering the fungal host comprising the promoter having modification in its transcriptional down-regulation mechanism.

4. The method according to any one of the preceding claims, characterized in that the promoter is selected from the group comprising a cellulase, a hemicellulase, an amylolytic enzyme, a hydrophobin, a protease, an invertase, a phytase, a phosphatase, a swollenin, a ligninolytic enzyme and a pectinase promoter.

5. The method according to claim any one of the preceding claims, characterized in that the promoter is selected from the group comprising cbh1 , cbh2, egl1, egl2, hfb1, hfb2, xyn1, swo, gla, amy, and pepA promoter.

6. The method according to any one of the preceding claims, characterized in that the modified region is located upstream of −162 of the Trichoderma cbh1 promoter.

7. The method according to any one of the preceding claims, characterized in that the modified region is located between the nucleotides −1031 and −162 in Trichoderma cbh1 promoter.

8. The method according to any one of the preceding claims, characterized in that the modified region is located between the nucleotides −1031 and −501 in Trichoderma cbh1 promoter.

9. The method according to any one of claims 1 to 8, characterized in that the modified region is located between the nucleotides −341 and −211 in Trichoderma cbh1 promoter.

10. A method for producing a fungal host for protein production, characterized in that the method comprises the steps of:
genetically modifying regulatory factors binding to the promoter of a gene encoding a secretable protein or mediating the regulatory signal in the expression of a selected secretable protein in a fungal host,
expressing the selected secretable protein in the modified fungal host in secretion stress conditions,
screening or selecting for cells showing enhanced or decreased protein expression of the selected secretable protein compared to the expression of the secretable protein in a non-modified host under the same conditions; and
recovering the fungal host.

11. The method according to claim 10, characterized in that the regulatory mechanisms are mediating the transcriptional down-regulation of the genes encoding proteins selected from the group comprising cellulases, hemicellulases, amylolytic enzymes, hydrophobins, swollenin, proteases, invertases, fytases, phosphatases, ligninolytic enzymes, and pectinases.

12. The method according to claim 10 or 11, characterized in that the regulatory mechanisms are mediating transcriptional down-regulation of the genes encoding proteins selected from the group comprising those encoded by the genes cbh1, cbh2, egl1, egl2, hfb1, hfb2, xyn1, swo, gla, amy, and pepA.

13. The method according to any one of claims 10 to 12, characterized in that the fungal host is a host obtained by the method of any one of claims 3 to 9.

14. The method according to any one of claims 10 to 13, characterized in that the regulatory factor is encoded by the ace1 gene and that the expression or activity of ace1 gene is reduced or abolished.

15. The method according to any one of claims 10 to 13, characterized in that the regulatory factor is encoded by the ace1 gene and that the expression or activity of ace1 gene is am plified or increased.

16. The method according to any one of claims 10 to 15, characterized in that the fungal host strain is selected from the group comprising Aspergillus ssp., Trichoderma ssp., Neurospora ssp., Fusarium ssp., Penicillium ssp., Humicola ssp., Tolypocladium geodes, Kluyveromyces ssp., Pichia ssp., Hansenula ssp., Candida ssp., Yarrowia ssp, Schizosaccharomyces ssp,, Saccharomyces spp.

17. The method according to any one of claims 10 to 16, characterized in that the strain belongs to Aspergillus ssp. or Trichoderma ssp.

18. The method according to any one of claims 10 to 17, characterized in that the strain belongs to A. niger or T. reesei.

19. A method for overproduction of homologous secretable proteins or production of heterologous secretable proteins in fungi, characterized in that the method comprises the steps of:
operable linking the promoter, obtained by the method of claim 1 or 2 or by the method of any one of claims 3 to 9, to the coding region of a gene encoding a selected secretable protein, said promoter being screened or selected on the basis of enhanced protein expression; and
expressing the selected secretable protein under the regulation of the promoter in a fungal host under suitable culture conditions; or
expressing the selected secretable protein in the fungal host obtained by the method of any one of claims 10 to 17, said fungal host being screened and selected on the basis of enhanced protein expression; and
recovering the protein product from the culture medium of the fungal host.

20. A method for decreased protein production of homologous secretable proteins in fungi, characterized in that the method comprises the steps of:
operable linking the promoter, obtained by the method of claim 1 or 2 or by the method of any one of claims 3 to 9 to the coding region of a gene encoding a selected secretable protein, said promoter being screened or selected on the basis of decreased protein expression; and
expressing the selected secretable protein under the regulation of the promoter in a fungal host under suitable culture conditions; or
expressing the selected secretable protein in the fungal host obtained by the method of any one of claims 10 to 17, said fungal host being screened or selected on the basis of decreased protein expression.

21. The method according to claim 19 or 20, characterized in that the protein product is selected from the group comprising proteins originating from bacteria or lower or higher eucaryotes or from fungal or mammalian origin, such as cellulase, hemicellulase, amylolytic enzyme, hydrophobin, protease, invertase, phytase, phosphatase, a ligninolytic enzyme, pectinase, immunoglobulin or tPA.

22. A method for optimised protein production of secretable proteins in fungi, characterized in that the method comprises the steps:
selecting a gene of a secretable protein,
operable linking the coding region of the gene encoding the selected secretable protein into a promoter not regulated by transcriptional down-regulation,
producing the selected protein under suitable culture conditions in a fungal host that overproduces proteins mediating down-regulation or produces these regulatory factors with enhanced activity; and
recovering the selected secretable protein from the culture medium of the host.

23. The method according to claim 22, characterized in that the promoter is obtained by the method of claim 1 or 2 or by the method of any one of claims 3 to 9 and screened or selected on the basis of enhanced protein expression.

24. The method according to claim 23, characterized in that the promoter is selected from the group of Trichoderma gpd1, cDNA1, ypt1, sar1, bgl2 promoter and Aspergillus gpdA promoter.

25. The method according to any one of claims 22 to 24, characterized in that the protein mediating down-regulation is ACEI.

26. A DNA sequence located between −1031 and −162 upstream of Trichoderma cbh1 promoter (nucleotides 1186 to 2053 in SEQ ID NO: 5) mediating transcriptional down-regulation of secreted proteins under secretion stress, or a DNA sequence having the same function in a promoter selected from the group comprising cbh1 , cbh2, egl1,egl2, hfb1, hfb2, xyn1; swo, gla, amy, and pepA promoter.

27. The DNA sequence according to claim 26, wherein the DNA sequence is located between nucleotides −1031 and −501 upstream of Trichoderma cbh1 promoter (nucleotides 1186 to 1714 in SEQ ID NO: 5).

28. The DNA sequence according to claim 26, wherein the DNA sequence is located between nucleotides −341 and −211 upstream of Trichoderma cbh1 promoter (nucleotides 1876 to 2004 in SEQ ID NO: 5).

29. Use of any of the DNA sequences of claims 26 to 28 in genetically modified form to enhance or decrease the expression of a selected protein in a fungal host in secretion stress conditions.


SEQUENCE LISTING (23 sequences)

SEQ ID NO: 1 - DNA, 1824 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaaga

SEQ ID NO: 2 - DNA, 2027 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagc

SEQ ID NO: 3 - DNA, 2003 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaag

SEQ ID NO: 4 - DNA, 1874 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgg

SEQ ID NO: 5 - DNA, 2053 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacgga

SEQ ID NO: 6 - DNA, 934 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggca

SEQ ID NO: 7 - DNA, 1014 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctc

SEQ ID NO: 8 - DNA, 1714 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgct

SEQ ID NO: 9 - DNA, 1474 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgt

SEQ ID NO: 10 - DNA, 1344 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtag

SEQ ID NO: 11 - DNA, 1184 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcg

SEQ ID NO: 12 - DNA, 1281 bp, Trichoderma reesei
ctcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 13 - DNA, 1031 bp, Trichoderma reesei
atgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 14 - DNA, 1201 bp, Trichoderma reesei
gacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 15 - DNA, 2215 bp, Trichoderma reesei
gaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtggcattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 16 - DNA, 501 bp, Trichoderma reesei
tgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 17 - DNA, 188 bp, Trichoderma reesei
cgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 18 - DNA, 211 bp, Trichoderma reesei
gaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 19 - DNA, 341 bp, Trichoderma reesei
gtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 20 - DNA, 391 bp, Trichoderma reesei
ttgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 21 - DNA, 162 bp, Trichoderma reesei
atgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 22 - DNA, 881 bp, Trichoderma reesei
tgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc

SEQ ID NO: 23 - DNA, 741 bp, Trichoderma reesei
aatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatc